(19) United States
(12) Patent Application Publication
(10) Pub. No.: US 2011/0111439 A1
(43) Pub. Date: May 12, 2011

(54) Elevation of Induced Heat Shock Proteins in Patient's Cerebral Spinal Fluid: A Biomarker of Risk/Onset of Ischemia and/or Paralysis in Aortic Surgery

(75) Inventors: James G. Hecker, Media, PA (US); Michael Lee McGarvey, Philadelphia, PA (US)

(73) Assignee: The Trustees of The University of Pennsylvania

(21) Appl. No.: 12/899,833

(22) Filed: Oct. 7, 2010

Related U.S. Application Data
(63) Continuation of application No. PCT/US09/02234, filed on Apr. 9, 2009.
(60) Provisional application No. 61/123,786, filed on Apr. 11, 2008.

(51) Int. Cl. G01N 33/53; G01N 33/566; C07K 14/47

(52) U.S. Cl. 435/7.92; 436/501; 530/350

(57) ABSTRACT

Provided are methods for intra-operatively predicting, detecting or diagnosing the risk or onset of spinal cord ischemia and/or associated permanent paralysis in a patient, based upon the stress-induced elevation of levels of heat shock proteins, specifically HSP70 and/or HSP27 in the cerebral spinal fluid of the patient, as measured during thoracic-aorta surgery, particularly thoracic aneurysm repair surgery, that will permit intra-operative medical intervention to try to prevent or attenuate severe, and often fatal, complications. Further provided are kits, assay devices and methods of analyzing biomarker data for use in pre-, intra- or post-operatively detecting the stress-induced elevations of the measured levels of HSP70 and/or HSP27, and the biomarker itself.

### CROSS-REFERENCE TO RELATED APPLICATIONS

This application is a continuation of International Application PCT/US09/02234 filed on Apr. 9, 2009 and published as WO 2009/126297 on Oct. 15, 2009, which claims priority to U.S. Provisional Application 61/123,786 on Apr. 11, 2008, each of which is incorporated herein in its entirety.

### GOVERNMENT INTEREST

This invention was supported in part by Grant No. R01-NS046591 (“Non-viral, controllable gene delivery for neuroprotection”) from the U.S. National Institutes of Health. The U.S. Government may therefore have certain rights in this invention.

### FIELD OF THE INVENTION

The present invention relates to minimizing a patient's risk of permanent paralysis when undergoing aortic surgery, by use of a biomarker to indicate a heightened risk of, or actual onset of, spinal cord or brain ischemia. In particular, the invention relates to intra-operatively detecting changed levels of stress-induced Heat Shock Proteins (HSP70 and/or HSP27) in the patient's cerebral spinal fluid.

### BACKGROUND OF THE INVENTION

Treatment of aortic aneurysms, including abdominal aortic aneurysms, thoraco-abdominal aneurysms, and thoracic aneurysms, involves surgical or endovascular repair in good-risk patients with aneurysms that have reached a size sufficient to warrant repair. There are essentially three surgical approaches, thoracic endovascular aortic repair (TEVAR), left atrium to femoral artery partial bypass (LA/FA), and deep hypothermic circulatory arrest (DHCA) with full cardiopulmonary bypass (CPB). Only LA/FA and DHCA are open procedures. However, while such surgery is effective from a macroscopic perspective, spinal cord ischemia with neurological deficit is a devastating complication that can occur during aortic aneurism repair, or paraplegia/paresis may present in the weeks following repair. Although endovascular repair is less invasive than conventional surgical repair, it is still associated with risk of spinal cord ischemia, manifested in a range of conditions from local neurological damage that self-resolves to permanent paralysis.

Despite clinical efforts to prevent spinal cord ischemia during thoracic aortic aneurysm (TAA) repair, paraplegia (paraparesis) remains a significant risk (Cina et al., J. Vasc. Surg. 40:36-44 (2004); Safi et al., Ann. Thorac. Surg. 67:1937-1939 (1999)). During surgical repair, clamps are placed across the thoracic aorta to prevent blood flow into the aneurysm. The aneurysm is opened to an area where the tissue is healthy, and sutures are applied to connect the healthy tissue to a synthetic fiber fabric graft. However, since blood flow to the spinal cord is jeopardized by the surgical repair, thoracic aorta aneurysm repair carries a relatively high rate of perioperative morbidity, including spinal cord ischemia, which occurs at a rate of between 5% and 21% of the patients. Any improvement in the ability to predict, detect, and intervene in the progression of ongoing spinal cord ischemia to prevent permanent damage would be a major advance and would be greatly aided by having a real-time, predictive biomarker for an elevated risk of paralysis. Ideally, measurement of such a biomarker during the surgical repair would allow the anesthesiologists, neurologists, and surgeons to modify or change the surgical procedure before the damage to cells becomes irreversible.

Heat shock proteins (HSPs) are inducible members of a highly conserved family of molecular chaperones, involved in a wide variety of roles in vivo, both physiological and pathological, and expression of the protein may be rapidly induced by severe physical or chemical stress stimuli. HSP27 and HSP70 are associated with cellular protection and recovery after a near lethal stress (Beere, J. Clin. Invest. 115:2633-2639 (2005); Da Rocha et al., J. Neurotrauma 22:966-977 (2005)). Examples of such stress stimuli include, but are not limited to: heat shock, cold, ischemia, anoxia and oxidative stress, glucose deprivation, and exposure to toxins, heavy metals, ultrasound and radiation (Kiang et al., Pharmacol. Ther. 80:183-201 (1998)). Assays to measure the levels of these proteins are well known to those skilled in the art.

Heat shock protein 27 (HSP27) is a member of the small heat shock protein family, which comprises members ranging from 15 to 30 kDa in size and which may be phosphorylated or oligomerized under various conditions. HSP27 is principally described as an intracellular chaperone capable of binding and stabilizing the actin cytoskelelton in response to stress. In addition, HSP27 can bind cytochrome c and prevent downstream caspase activation, making it a potent anti-apoptotic protein.

It has also been observed that HSP27 may be a potential biomarker for atherosclerosis, with expression of HSP27 diminishing with the progression of disease. (Martin-Ventura et al., Circulation 110:2216-2219 (2004)). Serum levels of HSP27 have been shown to be attenuated in patients with atherosclerosis compared to healthy age-matched, control individuals, and HSP27 may be involved in long term vessel wall homeostasis that is then lost with the progression of atherosclerosis. Moreover, the reduction in circulating HSP27 is reversed when patients are suffering from an acute coronary event, implying that HSP27 may be secreted into the extracellular space in response to cardiac ischemia. It appears to protect the vessel wall from stressful stimuli and prevents apoptosis, as well, which may be why HSP27 levels have been shown to be acutely increased in the serum following myocardial ischemia (Park et al., Circulation 114(9):886-893 (2006). It has been established that HSP27 expression in the vessel wall is lost as atherosclerosis progresses; thus, high levels of HSP27 in both the vasculature and circulation is likely athero-protective.

The rapidly inducible form of the 70-kDa heat shock protein (HSP70) is normally seen near the lower limit of detection by enzyme-linked immunosorbent assay (ELISA; ˜1 ng/ml) in the cerebral spinal fluid (CSF) of healthy humans, and typically remains at such low levels under normal conditions, including, for example, exercise to exhaustion (Dalsgaard et al., Exp. Physiol. 89:271-277 (2004)). CSF fluid surrounds, cushions and protects the brain and spinal cord from injury, and is usually collected for examination by a lumbar puncture procedure. However, because of the discomfort to the patient and other risks and difficulties involved in obtaining the CSF samples from patients, detection of infection or other disease states is typically performed on more readily available body fluids, such as blood, serum, plasma, urine, saliva or tears.

Numerous studies have used HSP70 immunohistochemistry as a marker for injury, for example, after cardiac surgery (Becker et al., J. Cardiovasc. Surg. 48:233-237 (2007); Dybahl et al., Eur. J. Cardio-Thorac. Surg. 25:985-982 (2004)) or in the central nervous system (CNS) (Chen et al., J. Neurotrama 15:171-181 (1998); Lindsberg et al., J. Cereb. Blood Flow Metab. 16:82-91 (1996); Mariucci et al., Neurosci. Lett. 415:77-80 (2007); Nowak et al., Brain Pathol. 4:67-76 (1994)). HSP70 also mediates cell protection, and homeostasis, and recent evidence suggests that HSP70 is important in the regulation of apoptosis and inflammatory responses, as well (Beere, J. Cell. Sci. 117:2641-2651 (2004)).

However, transcription and translation of these proteins increases dramatically in response to hypoxia or ischemia (Li et al., Sci. China C. Life Sci. 47:107-114 (2004); Nowak et al., Brain Pathol. 1994, supra). This serves as an endogenous mediator of intracellular protection, not just in the central nervous system, but in all tissues. Regner et al. (Da Rocha et al., J. Neurotrauma, 2005, supra) showed that elevated serum HSP70 levels, for example, predicted a poor outcome after severe traumatic brain injury in patients. Hart et al. (Carmel et al., Exp. Neurol. 185:81-96 (2004); Cizkova et al., Exp. Neurol. 185:97-108 (2004)) showed a robust induction of HSPs with 6 and 12 min of spinal cord ischemia in a microarray analysis. Contreras et al., Eur. J. Neurotrauma 22:966-977 (2005) demonstrated spinal cord protection using immediate ischemic pre-conditioning guided by somato sensory-evoked potential (SSEP) measurements, but HSPs were not measured as a part of the study.

Robinson et al., J. Neurosci. 25:9735-9745 (2005) showed an increase in HSP70 in response to ischemia in motoneurons, which reduced damage. They also showed that in vitro application of exogenous HSP70 to neuronal cells, conferred protection and improved long term motor neuron survival. In the CNS, multiple groups have also shown that the inducible heat shock proteins, in particular HSP70, confer neuro-protection in the brain from injury (Kelly et al., Curr. Med. Res. Opin. 18:55-60 (2002); Lee et al., J. Appl. Physiol. 100:2073-2082 (2006); Rajdev et al., Ann. Neurol. 47:782-791 (2000); Tsuchiya et al., Neurosurgery 53:1179-1188 (2003); van der Weerd et al., Exp. Neurol. 195:257-266 (2005)).

Nevertheless, until the present invention, there has remained a need in the art for a method of determining a patient's risk of permanent paralysis as a result of surgical repair of a thoracic aortic aneurysm, as predicted by a simple intra-operative biomarker. Moreover, a biomarker of spinal cord or brain ischemia that can be measured intra-operatively during the aneurysm resection repair process that could detect, diagnose or prognose the risk or onset of dangerously elevated levels of surgical ischemia, indicating severe cellular stress, before the damage to the cells is irreversible, would thus allow for the attenuation of this severe, and often fatal, complication.

### SUMMARY OF THE INVENTION

Heat Shock Proteins (HSPs) are released during severe cellular stress, thus their induction and the level at which they are induced in the CSF of a patient during aortic surgery, e.g., resection to repair an aneurism, provides a clinically useful biomarker brain and/or spinal cord cellular stress (ischemia) and subsequent neural damage resulting in patient paralysis.

Data is provided from experimental analysis of the levels of HSPs in the CSF from patients who were, at the time of the sample collection, undergoing thoracic aneurysm repair, as well as pre-operative and post-operative data related thereto. CSF samples were collected at regular intervals, and analyzed by immunoselection techniques to detect and quantify the concentration of stress-induced HSP27 and/or HSP70 in the CSF of the patient. Further comparisons were made to determine if the HSP27 and/or HSP70 levels have changed, in particular if they are elevated over a within-patient baseline or previous measurement of the HSP27 and/or HSP70 levels, or as compared with previously established control or normal levels. These results were correlated with intra-operative somatosensory evoked potentials (SSEP) and/or other standard intra-operative patient monitoring methods known to be used in aortic surgery. An identified elevation in the levels of the analyzed heat shock proteins in the patients was a biomarker, uniformly predictive, prognosticative or diagnostic of the risk or onset, respectively, of spinal cord ischemia and correlated with the likelihood of post-operative permanent paralysis in the patient.

It is an object of this invention to provide an understanding of the time course and a correlation with the expression and release of induced HSPs during brain and/or spinal cord cellular stress (ischemia), during and subsequent to thoracic-aorta surgery, wherein blood flow is blocked for some period of time, and an understanding of the role of the HSPs in cellular survival. It is also an object to provide a biomarker based upon changed levels of induced HSP levels, specifically HSP27 and/or HSP70, for determining risk of spinal cord or brain ischemia during the surgical repair and can detect the onset of dangerous levels of surgical ischemia by the levels of the biomarkers, prior to irreversible neural cell damage and/or permanent paralysis.

It is a further object of this invention to provide methods in which one or more within-patient measurements of the levels of stress-induced HSP27 and/or HSP70 in the CSF are made pre-, intra, and/or post-operatively in an aortic surgery to predict or diagnose those patients who are at greatest risk for paralysis during, or as a direct result of, aortic surgery, particularly thoracic aneurysm surgery. Preferably more than one such measurement is made to provide comparative induced HSP27 and/or HSP70 levels in the patient. In the alternative, or in conjunction with such within-patient comparisons, comparison may be made of the measurement as against a previously determined matched control.

It is another object of this invention to provide methods, based upon the stress induced elevation of the levels of HSP70 and/or HSP27 as measured in the patient during aortic surgery, that will permit intra-operative intervention to try to prevent or attenuate severe, and often fatal, complications, namely spinal cord ischemia and the associated risk of permanent paresis in the patient.

Surgically related, temporary paresis may resolve or be delayed post-operatively, and permanent paralysis may not develop in the patient until days or weeks after aortic surgery. Thus, it is another object of this invention to provide methods, wherein the CSF analysis is extended post-operatively to detect changes in HSP levels, or continued elevated levels of induced HSP (as compared with in-patient data or previously detected control levels), to predict risk of delayed post-operative permanent patient paralysis.

It is also an object of this invention to provide methods for predicted that there is little or no likely onset of spinal cord ischemia, and a low risk of post-operative patient paresis, when HSP27 and/or HSP70 in the CSF of the patient as measured intra- or post-operatively remain at within-patient or normal levels, nor is there a detected elevation of the measured levels.

It is also another object of this invention to provide kits, assay devices and methods of analyzing the biomarker data for use in predicting or diagnosing stress-induced elevations of the measured levels of HSP70 and/or HSP27 pre-, intra-, and/or post-operatively.

Additional objects, advantages and novel features of the invention will be set forth in part in the description, examples and figures which follow, and in part will become apparent to those skilled in the art on examination of the following, or may be learned by practice of the invention.

### BRIEF DESCRIPTION OF THE FIGURES

The foregoing summary, as well as the following detailed description of the invention, will be better understood when read in conjunction with the appended drawings. It should be understood, however, that the invention is not limited to the precise arrangements and instrumentalities shown.

FIGS. 1A and 1B show the average cumulative HSP70 concentration measurements as related to paralysis outcome at time points A through F, showing 95% confidence bars. FIG. 1A graphically depicts the data provided in the chart of FIG. 1B.

FIGS. 2A and 2B show the average HSP70 concentration measurements as related to paralysis outcome at time points A through F, showing 95% confidence bars. FIG. 2A graphically depicts the data provided in the chart of FIG. 2B.

FIGS. 3A and 3B show the average HSP70 concentration measurements as related to type of surgery (DHCA or LA/FA) at time points A through F, with 95% confidence bars. FIG. 3A graphically depicts the data provided in the chart of FIG. 3B. The p values are from a Kruskal-Wallis test of equal location parameters (i.e., medians).

FIGS. 4A-4D display side-by-side box-and-whisker plots of the independent variable distributions within the paralysis group of patients, as compared with the non-paralysis group for HSP70 with respect to each of the calculated variables listed in Table 2. FIG. 4A shows non-linearity (residual squared error); FIG. 4B shows maximum within-patient HSP70 concentrations; FIG. 4C shows the range of within-patient HSP70 concentration values; and FIG. 4D shows the average change in the HSP70 levels at pre- and post-time point (B). Thep values are from a Kruskal-Wallis test of equal location parameters.

FIGS. 5A-5D display side-by-side box-and-whisker plots of the independent variable distributions within the paralysis group of patients, as compared with the non-paralysis group for HSP27 with respect to each of the calculated variables listed in Table 2. FIG. 5A shows non-linearity (residual squared error); FIG. 5B shows maximum within-patient HSP27 concentrations; FIG. 5C shows the range of within-patient HSP27 concentration values; and FIG. 5D shows the range of HSP27 levels. Thep values are from a Kruskal-Wallis test of equal location parameters.

FIGS. 6A-6D display side-by-side box-and-whisker plots of the within-patient changes for HSP27 by paralysis outcomes. FIG. 6A shows change in HSP27 level from time point (B) to time point (C); FIG. 6B shows percent change in HSP27 level from time point (B) to time point (C); FIG. 6C shows average change is HSP27 level at pre- and post-time point (B); and FIG. 6D shows the percent change of HSP27 levels at pre- and post-time point (B). Thep values are from a Kruskal-Wallis test of equal location parameters.

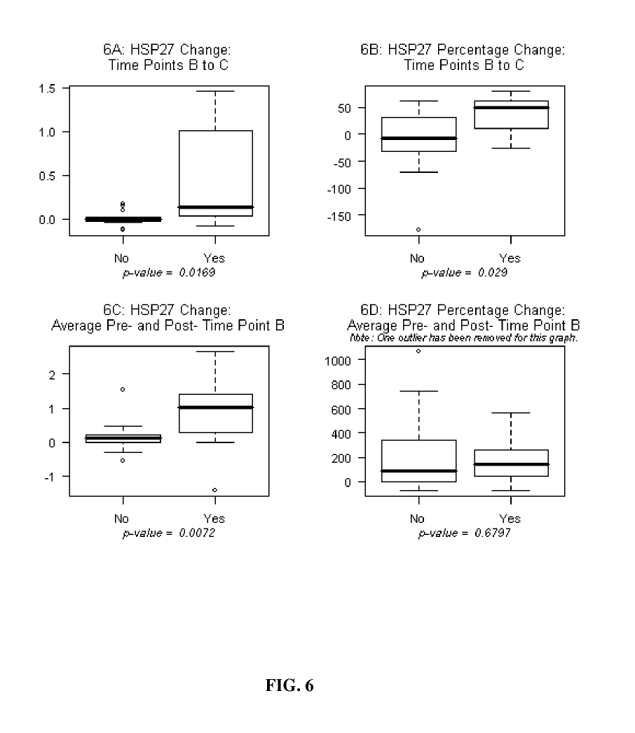

FIGS. 7A-7D graphically compare the paralysis and non-paralysis outcome groups with respect to four demographic variables: age (FIG. 7A), race (FIG. 7B), gender (FIG. 7C), and smoking history (FIG. 7D). Thep values are from a Kruskal-Wallis test of equal location parameters.

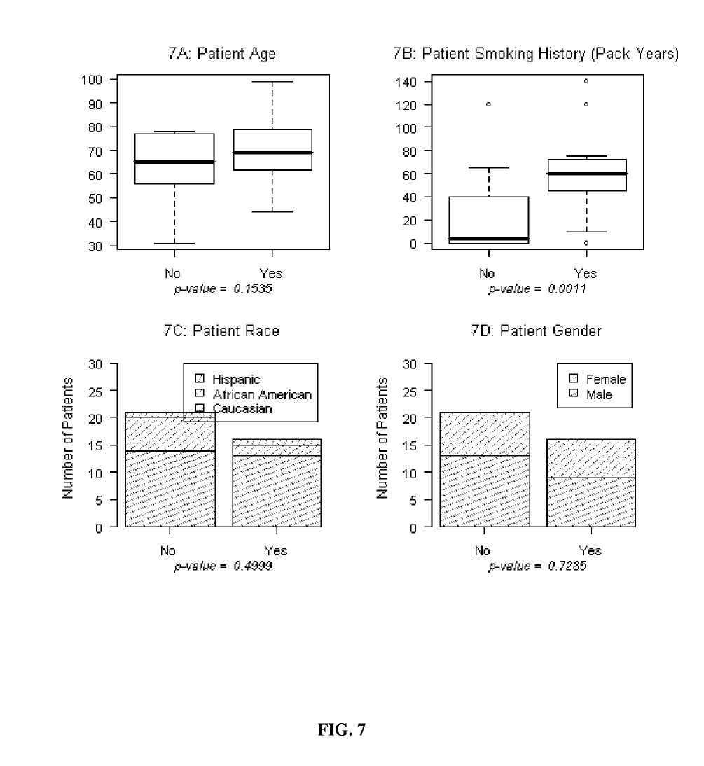

### DETAILED DESCRIPTION OF EMBODIMENTS OF THE INVENTION

Transcription, translation and expression of the inducible mammalian stress proteins, i.e., heat shock proteins, HSP27 and HSP70, are among the fastest of the intracellular severe stress responses in a patient, making their quantification an ideal biomarker indicative of, and predictive of, acute spinal cord ischemia of the patient during surgical aortic repair, e.g., repair of an aortic aneurysm, including abdominal aortic aneurysms, thoraco-abdominal aortic aneurysms (TAAA), and thoracic aneurysms (TAA). Thus, the present invention is based upon the determination that the increased quantifiable pre-, intra-, and/or post-operative levels of the stress-induced heat shock proteins HSP70 and/or HSP27 in the cerebrospinal fluid (CSF) of the patient, or changes in such levels, are “biomarkers,” indicative of the risk or severity of spinal cord ischemia and the potential for distal paralysis in the patient as a complication of the aortic repair surgery. Absence of change in the concentration levels is indicative of no apparent ischemia associated with the surgery.

During aortic resection, each patient generally receives a variety of intraoperative monitoring techniques, including CSF pressure monitoring, neurophysiologic monitoring with somato-sensory-evoked potentials (SSEPs), as well as transcranial motor-evoked potentials (tcMEPs), and monitoring of vital parameters for mean arterial pressure (MAP) and central venous pressure (CVP). The patients may also receive adjunctive pharmacologic therapy. However, the present invention was developed because previously no single direct measurement technique effectively recognized spinal cord ischemia as a predictor of permanent paralysis. “Ischemia” refers to a temporary deficiency of blood and oxygen in a portion of the body, often caused by a blockage in the blood vessel supplying that part. The brain and spinal cord are easily damaged by ischemia, specifically “spinal cord ischemia,” referring to too little blood reaching the neural network, which may rapidly result in irreversible cell damage.

Ischemia is generally referenced in terms of poor perfusion and, is therefore, distinguished from the concept of hypoxemia, meaning low oxygen content in the blood. While the end result may generally be the same, when ischemia is found to be present in the present case, it refers to spinal cord ischemia, as a direct result of the local loss of blood supply due to rupture or the mechanical obstruction of the aorta during surgery.

The present method of this invention for monitoring induced HSP27 and/or HSP70 levels in the CSF, and for determining induced HSP increases during open thoracic or thoraco-abdominal aortic surgery, offering a reliable intra-operative biomarker, indicating that ischemia may be causing pathologically relevant changes in the functional integrity of neural tissue, although the invention is not so limited. Use of elevated stress-induced HSP27 and/or HSP70 levels in the CSF during the surgery as a diagnostic tool, indicating increased risk or onset of potentially severe, and often fatal complications, permits anesthesiologists, neurologists and surgeons involved in the aneurysm repair process an opportunity to pre- or intra-operatively alter the surgical procedures, thus attenuating or remediating the onset of spinal cord ischemia and resulting paralysis.

Although evidence for the use of measured changes in the level of HSP27 and HSP70 is substantial and convincing, the HSPs are, nevertheless, a large, diverse, and fascinating family of molecular chaperones. Therefore, encompassed within the present invention are measured changes in levels of other HSPs, including, e.g., HSP90, HSP40, HSPA14, HSPA1A, HSPAIB, HSPA1L, HSPA2, HSPA4, HSPA4L, HSPA5, HSPA8, and/or HSPA9, to determine the effect of immediate stress as a response throughout the family of genes. Thus, other HSPs could offer additional or better responses for measuring the risk or onset of spinal cord ischemia, using, for example, the Luminex multiplex assay (Luminex Corp., Austin, Tex.) for determining different HSPs. Using a real time PCR or a PCR array for HSP (SA Biosciences, Frederick, Md.) to measure other HSPs at the messenger RNA level will further corroborate measuring HSP70 at the protein level.

Early intervention means that preservation techniques may be implemented, while the surgery is ongoing, to protect the spinal cord, thereby improving the neurological results while the physiologic situation can still be restored in an effort to prevent or at least minimize or attenuate patient paraplegia as a result of the identified spinal cord ischemia. Neuroprotective measures that may be taken in a patient undergoing aortic resection may include permissive moderate systemic hypothermia (34° to 35° C.) of the patient to reduce the oxygen demand of the neural tissue (5% per degree Celsius). In the event that the measured HSP27 and/or HSP70 biomarker increases, or if the CSF pressure exceeds 12 mmHg, CSF drainage is an additional tool to minimize spinal cord ischemia. The CSF may be drained to less than 10 mmHg to maximize the perfusion gradient. Also mean arterial pressure (MAP) may be increased, the epidural space may be cooled, or the intercostal arteries may be re-implanted. In addition, operative procedures may be immediately altered. If levels of the biomarker are too high in the patient, or if elevated pre-operatively, the surgical resection may even be canceled or at least modified, if the process is deemed to be too risky.

The term “biomarker” as used herein refers to target protein or polypeptide concentrations or concentration levels (“levels”) as determined in CSF test samples from the patient. Specifically, the biomarker of risk or onset of spinal cord ischemia during aortic surgery in accordance with the present invention is defined as the measurable change, e.g., elevation, of stress-induced HSP27 and/or HSP70 levels in the patient's CSF. HSP proteins or polypeptides include any fragments thereof, in particular, immunologically detectable fragments thereof. One of skill in the art would recognize that proteins or polypeptides released by cells of the central nervous system, and which become damaged during spinal cord ischemia, could become degraded or cleaved into such fragments. Thus, the term biomarker as used herein, may further include within the concentration detection methods, the detected levels of one or more fragments of HSP27 and/or HSP70 in the patient's CSF that may be detected as a surrogate for the biomarker itself, and used in an equivalent fashion to the mature HSP levels in the biomarker as applied in the methods described herein.

The term “sample” or “test sample” as used in this specification refers to a sample of bodily fluid obtained for the purpose of diagnosis, prognosis, or evaluation of a subject of interest, such as a patient. In certain embodiments, such a sample may be obtained for the purpose of determining the outcome of an ongoing condition or the effectiveness of a treatment regimen on a condition. As embodied in the methods of the present invention, the preferred test sample is CSF, but elevated HSP levels may also be found in the blood, serum, and plasma. In addition, one of skill in the art would realize that some test samples would be more readily analyzed following fractionation or purification procedures.

According to the invention, a diagnosis can be made on the basis of the detectable level of HSP27 and/or HSP70 in the patient's CSF during or after aortic resection surgery, which includes the presence of a polypeptide in a significantly lower or significantly higher amount with reference to a comparative (or control) test sample. In this invention, a “significant” change or elevation is any change which may be correlated with preventing permanent paralysis in a patient, but more specifically significantly refers to a change of 10%, 25%, 50%, 50%, 90%, or 100% (2-fold) or greater, extending to 200%, 300%, 400% or 500% or more, as the normal range for HSPs in the CSF are very low, even under exercise to exhaustion for example. Normal concentration levels of HSP values are 0 to 1.0 ng/ml in the Example that follows. Absolute values for HSP70 above 4 are problematic, meaning that a finding of an increase of 0.5× to 1×, to 2×-4× the baseline or normal within-patient level is a warning to the surgical team. Levels above that warn of extremely high risk of permanent damage.

However, for early detection, reliance is not limited to threshold levels, but rather early detection is based upon the development of a trend in the results, by analyzing multiple sequential related measures of the HSPs to determine whether elevation is sustained, or if it is a discrete outlier point, using the clinical utility using the step wise statistical analysis, involving data screening and statistical modeling disclosed herein. Characterization of the with-in patient HSP27 and HSP70 changes are provided in the Statistical Methods section in the Examples herein.

The term “diagnosis,” is used herein to identify a condition from a detectable set of existing marker values and/or patient symptoms. This is in contrast to disease or condition “prediction,” which is to predict the occurrence of disease before it occurs, and the term “prognosis,” which is to “predict” the risk of spinal cord ischemia and resulting paresis or its progression at a future point in time from one or more indicator value(s) at a previous point in time. In this case the elevation of stress-induced HSP27 and/or HSP70 levels in the CSF of a patient undergoing aortic surgery, e.g., aneurysm resection, or immediately following surgery if aortic flow was stopped for a period of time, operates as a biomarker indicating resulting spinal cord ischemia and possible complications. Thus, the appearance of the biomarker is a “diagnostic” tool, indicating increased probability (or “risk”) of spinal cord ischemia or the onset of potentially severe or fatal complications resulting therefrom.

The term “correlating,” as used in this specification, refers to a process in which a set of examples of clinical inputs from patients, such as biomarker levels, and their corresponding outputs, such as whether a subject is at increased risk of, or is suffering from, damaging spinal cord ischemia, are related to each other. This relationship can be determined by comparing such examples to levels of HSP70 and/or HSP27 in previously collected within-patient samples (e.g., a “pre-op sample”) or to samples from a control and/or non surgical population at a later point in time (“control samples”), and using the measured induced HSP level to differentiate between the samples as a function of time and circumstance, or in combination with certain probability levels. The selected biomarkers, each at a certain level range, which might be a simple threshold, are said to be correlative or associative with risk of, or onset of, spinal cord ischemia. Thus, the correlation of the patient's elevated, induced HSP27 and/or HSP70 levels during surgery, as compared with levels in the patient's earlier HSP27 and/or HSP70, e.g., a pre-op or pre-clamp sample, or with control samples, can be then be used as a biomarker for the prediction, prognosis, detection and/or diagnosis of spinal cord ischemia, as well as for the predicted risk and prognosis of its neurological outcome.

References to an increased or decreased induced HSP concentration, as compared a within-patient sample, or a control sample, do not imply that a step of comparing is actually undertaken, since in many cases, it will be obvious to the skilled practitioner that the concentration is abnormally high (increased or elevated over the comparative, matched sample) or low (less than the comparative matched sample).

Normal, otherwise healthy patients, particularly those under 30 or 40 years of age, have an HSP70 level in the CSF of ≦1 ng/ml. In the study presented in the Example section, intra-operative changes were seen in the HSP70 levels and the change acted as a biomarker correlated with the probability of permanent neurological spinal cord damage and paralysis as a result of the surgical processes, using the disclosed procedures. Because the measures are sequential and the second value is compared with the first, or with previous within-patient measurements, to determine whether the level has changed or elevated, and to what extent (or a measure as compared with a control), and at least two measures of HSP are needed. HSP70 and HSP27 concentrations are preferably each measured at each time point at which the CSF is sampled, but the levels are related and not completely independent of one another. Therefore, the biomarker correlation is predicted to underestimate the ability to prognose or diagnose risk of paralysis based on the disclosed statistical algorithms when there is more than one within patient measurement, or when there is at least one patient and a corresponding control measurement. One method for correlating the biomarker HSP27 and/or HSP70 levels is by running a feature selection algorithm and utilizing classification mapping functions described herein.

The biomarker is a diagnostic or predictive tool, which identifies trends in a patient. Its effect is not to determine whether the patient will definitively be affected by permanent paralysis following aortic surgery, as that offers no solution. Rather the purpose of the present biomarker is to provide a baseline, so the surgical team has an idea of the likely risks the patient will encounter during surgery, or to intra-operatively warn the surgical team of an elevated or increasing risk of impending neurological ischemia and permanent damage that is likely to result unless immediate medical intervention restores the oxygen to the deprived tissue.

Thus, the biomarker is a prognostic or diagnostic tool, but of paralysis only if the ischemia is not attenuated. The biomarker is intended to function as an early warning system, to intra-operatively alert the medical team that rapid action must be taken to resolve ischemia, an otherwise invisible problem during the surgery, that prior to the present invention remained unrecognized until after irreversible neurological damage had already been done.

Assuming, therefore, a pre-operative or normal within-patient HSP70 level in the CSF of a patient is ≦1 ng/ml the following levels of change determined intra-operatively in the patient during aortic surgery, e.g., repair of a thoracic aortic aneurysm, function as a biomarker to the surgical team of the probable or expected corresponding increase in the risk of permanent paralysis if the ischemia remains untreated. The following values are presented as an estimate, with substantial variation between patients and may vary ±10% (referred to as “about”), but it is provided to give numerical value to the trends determined by the use of the changed/increased HSP levels as biomarker, predictive of risk or diagnostic of onset of spinal cord ischemia. The correlated probabilities assume that the patient presents no other confounding factors, such as old age, long smoking history, or illness such as diabetes, although in many such surgeries a variety of patient factors create variables, as shown in the Tables presented in the Example section.

If the HSP70 level remains ≦1 ng/ml CSF, the patient's risk of paralysis if untreated would remain about 10% or less. An increase from ≦1 ng HSP70/ml CSF, to about 1-2 ng HSP70/ml CSF, would increase the patient's risk of paralysis if untreated to about 12% or less. An increase from ≦1 ng HSP70/ml CSF, to about 2-3 ng HSP70/ml CSF, would increase the patient's risk of paralysis if untreated to about 15% or less; to about 3-4 ng HSP70/ml CSF, would raise the risk to about 18%; to about 4-5 ng HSP70/ml CSF, would raise the risk to about 25%. An increase to about 5-6 ng HSP70/ml CSF, would raise the risk to about 35%; to about 6-7 ng HSP70/ml CSF, would raise the risk to about 45%; to about 7-8 ng HSP70/ml CSF, would raise the risk to about 55%. An increase to about 8-9 ng HSP70/ml CSF, would raise the risk to about 65%; to about 9-10 ng HSP70/ml CSF, would raise the risk to about 75%, and about 10 or more ng HSP70/ml CSF, would raise the risk to about 85% or higher. The same trends and proportions are seen in changes in HSP27 levels.

Even if confounding factors are present the percent of increased risk of permanent paralysis remains directly correlated to the elevation measured in the biomarker, but likely at a higher starting level of HSP in the CSF. If the patient's HSP levels were already high when measure pre-operatively, e.g., higher than ≦1 ng HSP27 or HSP70/ml CSF, and if the decision were made to continue the surgery, the risk of paralysis as a result of the surgery would already be elevated over the otherwise normal levels. For example if the patient's HSP70 were measured pre-operatively to be 3.5 ng/ml CSF, then that person would already have about 18% risk of paralysis as a result of the aortic surgery, even if no additional HSP were induced intra-operatively. However, a two-fold increase to about 7 ng/ml CSF would immediately provide the surgical team with a biomarker warning that the risk of paralysis has elevated to >50%, unless intervention is provided to alleviate the spinal cord ischemia.

As used herein, “changed” or “altered” levels of HSP27 and/or HSP70 refer to elevated, increased or reduced levels, using standard dictionary meanings. As previously noted, “levels” refer to the measured concentration or quantity of HSP27 and/or HSP70 in the CSF at the time the sample is drawn. Particularly relevant to the present method are either “no change” or minimal change, or “elevated” or “increased” levels, when the above correlating or comparative steps are applied. Increased expression of HSP27 and/or HSP70 is also up-regulation. “Elevated” or “increased” levels assumes the standard meaning of the terms, and herein specifically refers to a statistically significant higher concentration of HSP27 and/or HSP70 in the CSF than was present in the patient before aneurysm surgery, or prior to aortic clamping in the patient, or as compared with control samples. Such increased expression or up-regulation of HSP27 and/or HSP70 indicates probable spinal cord ischemia or risk thereof. Generally, increased levels of induced stress protein as a biomarker in the CSF of the patient undergoing aortic aneurism repair surgery, rise directly with the risk of ischemia and resulting permanent paralysis.

The risk of ischemia or paralysis of the patient during or resulting from aortic surgery, specifically TAA or TAAA, is “heightened” or statistically significant if it is at least 5% or 10% or 20%, or at least 50%, or even more at least 80% or greater up to 100% as compared with the risk of spinal cord ischemia and/or paralysis before the surgery. While the patient's risk of paralysis going into the surgery is the same whether HSP is measured or not, however, the predicted risk of spinal cord ischemia and/or paralysis as predicted by a change in the level of HSP 27 and/or HSP70 in the patient's CSF is the determinative value in this invention. If measured pre-operative HSP levels are elevated over normal or expected values, then that is an indication that the patient has relatively less functional reserve than would be expected in a normal patient, meaning that patient can tolerate little to no reduction in spinal cord blood flow without the onset of ischemia, and such a patient is, therefore, at higher risk (>50% higher) of paralysis if aortic resection surgery is performed. A pre-operative elevation of a patient's HSP27 and/or HSP70 levels in the CSF by ≧50% over normal healthy adult levels, is considered to be high, and may act as a threshold cause for changing surgical procedures or canceling the surgery. If ischemia is detected intra-operatively by the disclosed change or elevation in HSP levels, then at that point the patient's risk of paralysis may be considered to be increased to ≧70%, unless the surgical team does something to restore oxygenated blood flow to the neurological tissue or otherwise preserve the spinal cord and function.

The patient's risk of rupture of the aneurysm without the surgery is not factored into this calculation.

By “reduced” is meant a lessening or reduction of the biomarker or levels of the biomarker present in the CSF. A change in the surgical procedure as a result of the quantified or comparative level of to remedy or rectify the detrimental effect of spinal cord ischemia is referred to as “protecting” the patient.

The patient of this invention is preferably a human patient, however, it can be envisioned that the presently described methods of the present invention may be applied to any animal undergoing surgical resection of the aorta, particularly to resolve an aortic aneurysm. Although described herein in terms pf predicting, prognosticating, detecting or diagnosing spinal cord ischemia and risk of paralysis in connection with aortic resection to repair an aneurysm, the present invention further encompasses such use at any time aortic blood flow is stopped for a period of time during medical intervention for any purpose, and is not limited to resection surgery.

As used herein, the terms “treating” and “treatment” in response to the identified biomarker increase are intended to include the terms “attenuating,” “preventing” and “prevention” of spinal cord ischemia, as well as paralysis resulting from the spinal cord ischemia. “Attenuating” means any intervention, particularly intra-operative intervention, which affects the onset of permanent patient paralysis. “Preventing” refers to effectively 100% levels of prophylactic inhibition of permanent paralysis, but that may not mean the there is a 100% protection from spinal cord ischemia, only that it would no longer result in permanent paralysis.

Neurophysiologic monitoring has become routine during thoracic and thoraco-abdominal aortic repair because it is easy to implement and does not hinder peri-operative and/or intra-operative activity. Intra-operative neurophysiologic monitoring offers traditional techniques that may still be implemented in conjunction with the biomarker monitoring steps of the present invention. The functional integrity of the spinal cord can be further evaluated by measuring somatosensory-evoked potentials (SSEPs), as well as transcranial motor-evoked potentials (tcMEPs), which exhibit a change upon induced ischemia. Loss of tcMEP and SSEP is associated with spinal cord ischemia, and patients exhibiting tcMEP and/or SSEP loss have a higher risk for paraplegia or death than patients without such loss.

TcMEPs are induced to observe signal transmittance in the descending neuronal motor pathways. Upon transcranial stimulation of the motor cortex, a motor response occurs in the peripheral muscles. SSEPs assess the function of the ascending neuronal sensory pathways. The cerebral response is measured continuously after electrical stimulation of a peripheral nerve. Both the tcMEP and SSEP monitoring methods assess spinal cord function and have a complementary controlling character: tcMEP recordings reflect the functional integrity of the anterolateral cortico-spinal motor tracts, and SSEP recordings monitor the posterior sensory tracts of the spinal cord.

However, the tcMEP and SSEP measurements when used alone, each have their drawbacks. The basic SSEP recording involves signal averaging to minimize the “noise” in the reading, requiring the responses of 100-1000 stimuli to be averaged to resolve these electrical artifacts. The process involves comparisons between intraoperatively gained potentials and the patient's individual baseline SSEP values to enable a neurophysiologic monitoring team to assess acute spinal cord function. However, reliance upon SSEP measurements requires obtaining baseline recordings from the patient before surgery, because interference from technical equipment may affect the viability of the intraoperative measurements.

Moreover, a distinction must be made regarding the relevance and prognostic value of tcMEP loss compared with a SSEP loss. The tcMEPs allow insight into spinal cord function within several minutes after an intervention, and the tcMEPs can be re-measured after only a short interim. The SSEPs, on the other hand, gradually deteriorate and exhibit a retarded restoration period and an impending long-term loss, even after an intervention to counteract any potential malperfusion. Both types of evoked potentials gauge different anatomic spinal cord structures, each having a different vascular supply.

Despite their recognized drawbacks, traditional methods have implemented both of these monitoring methods, even though SSEP measurements are limited. However, when used in conjunction with other methods, including with the methods of the present inventions, the traditional techniques provide additional safety, as well as additional diagnostic capabilities and fail-safe mechanisms. Moreover, the reversibility of changed potentials, as a result of traditional neurophysiologic monitoring methods to coincide with an uneventful neurological outcome, when they have occurred, demonstrates that if an impeding spinal cord ischemia is diagnosed by methods of the present invention, the neurological deficit can still be corrected at a reversible stage before resulting in paralysis.

Other traditional neuroprotective measures have included adjusting the mean arterial pressure (MAP) to more than 80 mmHg by the use of, e.g., noradrenaline, and decreasing and central venous pressure (CVP) to less than 12 mmHg by use of, e.g., nitroglycerine and restrictive volume management to restore spinal cord perfusion pressure. A CSF drain may be inserted well in advance of surgery, e.g., by 1-12 hours before surgery, for two main reasons. First, it may be necessary to register a baseline of the patient's individual CSF pressure under healthy or pre-operative conditions, thereby permitting an aberrance of pressure in a pathologic situation to be reliably detected. Secondly, it also permits a controlled insertion, as opposed to a late insertion in response to a neurological emergency or complication. Infections and consecutive meningitis have not been a significant problem with early introduction of the CSF drain, which simplifies collection of samples for quantifying the HSP27 and/or HSP70 levels in patients undergoing endovascular thoracic and thoraco-abdominal aortic repair if the CSF drain is already in place. Routine CSF pressure monitoring may be done for 1, 2 or 3 post-operative days, and if CSF pressure exceeds 12 mmHg, CSF may be drained again under pressure control.

Although it is assumed that induced HSP levels in the CSF reflect spinal cord ischemia because of the type and location of the aneurism repair surgery, HSPs are induced in any tissue that is exposed to a near-lethal stress. Elevated HSPs in the CSF could also be due to brain ischemia, or due to increased systemically circulating levels, if the blood brain barrier (BBB) is not intact. For example, in the present study only one patient exhibited an initial HSP70 level significantly greater than 2.0 ng/ml CSF at the first time point, but a carotid endarterectomy (CEA) had been performed on this patient the previous day, and her baseline neurological exam was normal. As a result, it is recognized that the significance of the HSP levels as a biomarker in this process is relative. It is the intra-operative change to an elevated level above the baseline measurement that is predictive of neurological ischemia, whether it is of the brain or the spinal cord, providing the surgical team with a warning of possible patient paresis while corrective measures can be taken.

In one embodiment, the present method relies upon recurring measurements of HSP27 and/or HSP70 in the CSF of the patient at multiple time points to assure that a significant increase in the induced HSP is not representative of an isolated, aberrant time point. Recurring measurements may be made beginning as early as 12 hours prior to surgery, throughout the surgery, and continuing for 1-3 days postoperatively, although it is noted that the permanence of paralysis resulting from cell damage caused during that time frame may not be known until weeks after the surgery. In another embodiment of the invention, if recurring measurements are not made, or not possible, the use of a single CSF sample, while not optimal, may be used to quantify the induced HSP 27 and/or HSP70 levels, as compared with previously collected or established control data to detect, predict, prognose or diagnose the probable onset of spinal cord ischemia.

The present data support the finding that not only is a significant expression of HSP27 and/or HSP70 induced when a patient's spinal cord is exposed to ischemia, but using elevation of the stress-induced HSP levels is an effective biomarker for predicting permanent post-operative paraplegia if the ischemia remains untreated. Moreover, methods using the elevated levels of measured induced HSP as a predictive biomarker appear to be more sensitive to the effects of the aneurism repair surgery than traditional SSEP measurements in which changes (specifically those changes scaled as 2-6) are directly correlated to central signs of sensory deficiency.

While SSEP changes have traditionally served as surrogates for motor compromise stemming from central ischemia, use of the information relating to the HSP changes in accordance with the present invention is a significant improvement, simplifying diagnosis and prediction of spinal cord ischemia in a patient as an accepted standard of care to prevent paralysis. The present data demonstrate that a greater percentage of patients with increased induced HSP levels and/or persistently elevated HSP27 and/or HSP70 levels became paraplegic, as compared with patients in which only SSEP changes were identified.

In one embodiment of the present invention, therefore, methods are provided for predicting a patient's risk of spinal cord ischemia pre-, intra- or post-operatively in a patient wherein the surgery is thoracic-aorta resection, particularly involving aortic cross-clamping, but not limited thereto. Because the recognized risk of permanent paralysis is high in the patient in the case of spinal cord ischemia, the biomarker prediction is also a prognosis of the risk of permanent paralysis following the surgery, when no paralysis was present before the surgery. The method relies upon the detection of changed or elevated levels of stress-induced HSP27 and/or HSP70 in the CSF of the patient as measured over a time course pre-, intra- or post-operatively, as compared with either control samples, a profile of known HSP27 and/or HSP70 concentrations in CSF, and/or with the level of the protein drawn from the patient pre-operatively as a “within-patient baseline” value.

In another embodiment of the present invention, methods are provided for detecting the onset of spinal cord ischemia intra- or post-operatively in a patient who is undergoing or has recently undergone the above described aortic resection surgery. Spinal lumber drains, through which CSF can be sampled, typically are in place for approximately 48 hours in neurologically intact surgical patients. In case of patients who are unconscious, high risk, who have had a transient spinal ischemia, or have had a new drain placed because of recurrent ischemia, which has occurred after the initial drain has been removed, may stay in place indefinitely for therapeutic and sample collection use. Sample time intervals before and after application of aortic cross clamp, are as described, e.g., 15, 20, 30 or 60 minutes for the first 1, 2, 3, or 4 hours, followed by a lengthening of the duration between sapling intervals to 1 or 2 hours. This sampling frequency provides warning of a need for both treatment, intervention (medical, critical care, and/or surgical), as well as determination whether the paralysis is transient and reversible, or permanent.

Of course, applying the method of measuring the SSEP changes and the present method of monitoring HSP27 and/or HSP70 levels, in combination, offers an alternative embodiment of the present invention. Any traditional method for detecting neurological changes in the patient may be used in conjunction with the present invention.

In the post operative period, SSEP levels in the patient are not monitored, but HSP27 and/or HSP70 levels can be measured until removal of the lumbar drain. Post-operative measurements of induced HSP27 and/or HSP70 levels may continue for 1, 2, 3, 6, 12, 24, 36, 48 or 72 hours, or more, following reperfusion of the aorta after the cross-clamp is removed, or for so long as the lumbar drain remains in place. Frequent samples, drawn every 20 or 30 minutes immediately after removal of the aortic cross clamp for 1 or two hours would be expected, and then if there has not been a detection of spinal cord ischemia, the time interval between samples can be lengthened to every 60 or 120 minutes for 6 to 12 hours. This frequency appears to provide at least one critical threshold for determining impending “irreversible spinal cord ischemia.” A combined calculation of magnitude of induced HSP elevation and duration of increased HSP is highly predictive of impending irreversible ischemia. In yet another embodiment of the present invention, therefore, methods are provided for detecting the risk or onset of post-operative spinal cord ischemia and/or probability of permanent paralysis in a patient who has recently undergone the above described aortic surgery.

The percentage of patients who developed paraplegia was comparable in both groups of open aortic repairs (4 out of 10 deep hypothermic circulatory arrest (DHCA) patients, and 10 out of 20 left atrium to femoral artery partial bypass (LA/FA) patients). However, the mean HSP27 and/or HSP70 levels at successive time points in patients with plegia after DHCA, were considerably higher than the measured levels of HSP27 and/or HSP70 in patients with plegia after LA/FA, especially in the 12 and 24 hours post cross-clamp. This trend was seen both in patients with paraplegia, as well as in patients who had no postoperative neurological complications. It is unclear whether DHCA has a greater degree of ischemic injury than LA/FA, whether this is brain or spinal cord injury, and whether these patients are thus afforded protection from even greater injury by the induction of hypothermia. It is possible that the DHCA patients were suffering a subclinical neurocognitive injury that has been associated with full cardiopulmonary bypass (CAP). LA/FA patients, who do not undergo CAP bypass (typically 32° C. is maintained during CAP bypass), are kept warmer during the operation.

Regardless of the cause, LA/FA patients not only had paralysis at lower mean HSP27 and/or HSP70 levels, but also appeared to be at greater risk of permanent neurological injury. Two out of the four patients with neurological compromise after DHCA surgery recovered fully in the Example that follows, whereas only two of the ten patients in the LA/FA group with similar complications recovered even partial function following intervention. The HSP27 and/or HSP70 level elevation noted in DHCA patients may represent a significantly higher level of ischemia resulting from this operative approach, due to a reliance on retrograde blood flow, plus cardio-pulmonary bypass to provide cerebral perfusion. In the alternative, the elevation induced in the HSP27 and/or HSP70 levels may reflect systematical increased expression of the biomarker in DHCA surgeries, because hypothermia, as well as hyperthermia, is known to induce HSPs. DHCA patients may reflect more extensive or complicated repairs and may be exposed to a double or triple insult: severe hypothermia, complete cessation of blood flow to the CNS, and simultaneous cardiovascular bypass (although the interaction of cold, reperfusion, and Coronary Artery Bypass Graft (CABG) surgery is poorly understood).

Neurophysiologic Monitoring of Induced HSP27 and/or HSP70 in the CSF. Methods for the collection of CSF samples from a patient are well-known to one of ordinary skill, but will be described in general. A CSF drainage catheter was placed into all patients in this study. The CSF drain was routinely inserted preoperatively by the anesthesiologist the day of surgery. Insertion was performed using standard protocols by puncture, preferentially between the spinous processes L4/5 at the level of the posterior iliac crest in the left lateral decubitus position with a 14-gauge Tuohy needle. Once the CSF began to flow, a lumbar drain, e.g., a Portex lumbar drain (Portex Ltd, Kent, UK) was inserted ˜30 cm into the intradural space. Correct placement was checked by observing the spontaneous flow through the catheter.

The drain was then blocked, secured at the puncture site with sterile dressing, and the distal blocked connector was led along the right lateral abdomen and chest and secured in the anterior auxiliary line. The drain was secured with wide tape dressing along its entire course and connected to the external monitoring equipment before surgery.

Preferably, a patient baseline CSF sample was drawn at the time of lumbar drain placement, prior to surgical incision, although this is not always possible. The patient's history and condition was also evaluated. Individuals exhibiting no symptoms of paresis, e.g., stroke, operated as controls in the present Example section, although they may have other conditions or diseases causing their CSF to be otherwise collected, and paresis could occur in the control patients at a later time. CSF samples were collected from the control patients, and assayed for the presence of HSP27 and/or HSP70. If a measurable level of induced HSP was present in a control sample, the concentration level was recorded.

The patient history and clinical information, such as, but not limited to, age, race. sex, gender, medical history, time from onset of symptoms to treatment, NIHSS score, biochemistry and vital signs at admission presentation status (emergent versus scheduled), and neuroimaging findings, whether deep hypothermic circulatory arrest (DHCA) was induced intraoperatively, whether intercostal vessels were re-implanted, presence of dissection or contained rupture, the repair type [cardiopulmonary bypass (CPB) with DHCA, left atrium/femoral artery bypass (LA/FA), transthoracic endovascular repair (TEVAR)], cross-clamp time, circulation arrest time, bypass time, maximal change in intraoperative MAP, and extent of repair (collectively referred to herein as the “patient characteristics”) were collected and recorded.

Intraoperatively, CSF was drained if the CSF pressure exceeded 12 mmHg. Other neuroprotective measures included adjusting the MAP and CVP. Routine CSF pressure monitoring was done until the third postoperative day. If CSF pressure exceeded 12 mmHg, CSF was drained again under pressure control. CSF samples were drawn from the patient during surgery at recurring intervals, which may or may not be regular intervals. Preferred time periods for sampling the CSF during the operation, are at 15 minute or 20 minute intervals, but 5 minute, 10 minute, 30 minute 45 minute, and 60 minute intervals are also permitted, as well as longer times, e.g., once an hour or two, or only at the time points indicated in the Example section. The timing of the collection is optional to the surgical team, since the changed levels of HSP are not dependent on when the collection is made, but rather on the condition of the patient. The timing of the collection only changes the likelihood of identifying the change as soon as possible in the patient.

Time is measured at the time of, or from the beginning of, the surgical process, at the time of application of the aortic cross-clamp, at the time removal of the cross-clamp, at the time perfusion into the aorta initiates after removal of the cross-clamp, at the time of onset of symptoms of spinal cord ischemia, or at the time of determination by any neurological monitoring method of the onset of spinal cord ischemia. The most critical times during surgery for measuring induced HSP27 and/or HSP70 levels in the CSF is at the time of application of the aortic cross-clamp, at the time removal of the cross-clamp, and at the time perfusion into the aorta initiates after removal of the cross-clamp. Typically after the baseline measurements have been established, intraoperative measurements of induced HSP70/27 levels are made in CSF samples drawn at 15, 20, or minute intervals throughout the surgery, and then the measurements are made post-operatively at about 60 or 120 minute intervals, or more. The most critical times for CSF sample collection are in the immediate minutes and hours after cross clamp of the aorta or after the stent is deployed in TEVAR.

One recognized therapeutic intervention during aortic surgery is to increase the systemic blood pressure if spinal cord ischemia is suspected to attenuate the risk of patient paralysis, but such increased blood pressure can itself be detrimental to the patient, meaning that a biomarker would be useful to determine the safety of such intervention. Consequently, it is another embodiment of the present invention to apply the biomarker methods defined herein to determine if the levels of stress-induced HSP27 and/or HSP70 in the CSF during surgical intervention confirm that the patient's cellular stress level is acceptable to withstand methods that will increase the blood pressure. Recognizing that the HSP concentrations in the patient's CSF are at normal levels, as compared with control data, or that there is no significant intra-operative change or elevation in the level of induced proteins may potentially be just as important as detecting injury or probable spinal cord ischemia. Such a determination permits the medical team to better understand the patient's status and to proceed with an added measure of safety, permitting the regulation of the patient's blood pressure within acceptable, non-damaging levels.

Thus, the biomarker levels and the patient's clinical information in the present methods form a dataset, at which each time point of CSF sample collections, pre- intra- or post-operatively, are evaluated and processed to provide “data outputs relevant to the clinical outcome of interest,” which is spinal cord ischemia or risk thereof, and the resulting paresis occurrence or non-occurrence. As previously indicated, the induced HSP levels are relevant, and therefore, must be evaluated in terms of within patient measurements and condition. The control samples provide supporting information and a numerical basis against which the patient samples are compared to provide added safety to the patient and to confirm the acceptability and reasonability of the measured patient calculations.

Immunoselection Techniques. Any known method of immunoassay may be used to detect the stress-induced elevation of the HSP27 and/or HSP70 levels in the CSF samples, wherein reference to “detecting” a polypeptide should be understood to include a reference to compositions and methods for detecting to quantitative variations of HSP27 and/or HSP70 in the CSF. Such immunoassays include, e.g., enzyme-linked immunoassays (ELISA), radioimmunoassay (RIAs), competitive binding assays, and the like. Specific immunological binding of the antibody to the marker can be detected directly or indirectly.

In indirect assays, such as in a sandwich assay, an antibody (e.g., polyclonal) to the polypeptide is bound onto a variety of solid supports, such as magnetic or chromatographic matrix particles, the surface of an assay place (such as microtiter wells), pieces of a solid substrate material (such as plastic, nylon, paper), and the like, and incubated with the sample and with a labeled second antibody specific to the polypeptide to be detected. An assay strip could be prepared by coating the antibody or a plurality of antibodies in an array on solid support. This strip could then be dipped into the test sample and then processed quickly through washes and detection steps to generate a measurable signal, such as a colored spot. Alternatively, an antibody capture assay can be used, wherein the test sample is allowed to bind to a solid phase, and the anti-polypeptide antibody (polyclonal or monoclonal) is then added and allowed to bind. If a polyclonal antibody is used in this context, it should desirably be one which exhibits a low cross-reactivity with other forms of polypeptide. After washing away unbound material, the amount of antibody bound to the solid phase is determined using a labeled second antibody, anti- to the first.

A direct assay can be performed by using a labeled anti-polypeptide antibody. The test sample is allowed to bind to the solid phase and the anti-polypeptide antibody is added. After washing away unbound material, the amount of antibody bound to the solid phase is determined. The antibody can be labeled directly rather than via a second antibody.

Alternately, in a competition assay, performed between the sample and a labeled polypeptide or a peptide derived therefrom, the two antigens are in competition for a limited amount of anti-polypeptide antibody bound to a solid support. The labeled polypeptide or peptide can be pre-incubated with the antibody on the solid phase, whereby the polypeptide in the sample displaces part of the polypeptide or peptide thereof bound to the antibody. In another embodiment, the two antigens are allowed to compete in a single co-incubation with the antibody. After removal of unbound antigen from the support by washing, the amount of label attached to the support is determined and the amount of protein in the sample is measured by reference to standard titration curves established previously.

Direct labels include fluorescent or luminescent tags, metals, dyes, radionuclides, and the like, attached to the antibody. Indirect labels include various enzymes well known in the art, such as alkaline phosphatase, horseradish peroxidase and the like. As disclosed herein, the label is preferably an enzyme. The substrate for the enzyme may be color-forming, fluorescent, chemiluminescent or electrochemical, and can be soluble or precipitating. Alternatively, the label may be a radioisotope or fluorescent, e.g., using conjugated fluorescein. The enzyme may be conveniently used colorimetrically, e.g., using p-nitrophenyl phosphate as a yellow-forming substrate with alkaline phosphatase. Such procedures may be mechanically processed, e.g., using a RAMP Biomedical device, called the Clinical Reader', which uses the fluorescent tag method, though the skilled artisan will know of many different machines and manual protocols to perform the same assay to determine analyte concentration.

For a chemiluminescent assay, the antibody can be labeled with an acridinium ester or horseradish peroxidase. The latter is used in enhanced chemiluminescent (ECL) assay. Here, the antibody, labeled with horseradish peroxidase, participates in a chemiluminescent reaction with luminol, a peroxide substrate and a compound, which enhances the intensity and duration of the emitted light, e.g., 4-iodophenol or 4-hydroxycinnamic acid.

In the exemplified embodiment, an ELISA test is used to detect the polypeptide. ELISA defines a well-known assay, for which kits are commercially available. ELISA techniques were typically used herein to determine the presence of, or to quantify the levels of, HSP27 and/or HSP70 in the collected CSF samples. An ELISA is a primary binding, sensitive immunoassay that uses an enzyme-labeled immunoreactant and an immunosorbent, specifically an enzyme linked to an antibody or antigen as a marker for the detection of a specific protein, namely an antigen or an antibody. Either antigen or antibody is bound to a solid substrate (polystyrene surface), and a second antibody to which enzyme is conjugated is added, followed by a substrate for the enzyme. ELISA techniques offer low cost, simpler equipment, faster ‘turn-around time,’ and avoid problems inherent in handling radioactive substances. Results can currently be provided by an ELISA test in under 4 hours, but more rapid tests are preferred, providing HSP levels within ≦3 hours, ≦2 hours, ≦1 hour, ≦30 mins, ≦15 mins, or instantaneously as with a dip stick type test.

In an ELISA assay, labeling is done by covalently binding the enzyme to the test substance through an enzyme-protein coupling agent, such as glutaraldehyde. When used for the in vitro determination of HSP27 and/or HSP70 concentrations in the CSF samples, the ELISA tests are nearly as sensitive as radioimmunoassay, and more sensitive than complement fixation, agglutination, and other techniques. Increases or decreases in biomarker levels, as well as de minimus change or the absence of change in biomarker levels, provides useful information about the patient status that includes, but is not limited to identifying the presence or absence of an adverse, event, such as spinal cord ischemia, approximate time from onset of the adverse event, the presence and amount of salvageable tissue, the appropriateness of intervention therapies, the effectiveness of various therapies, identification of the severity of the adverse event, and identification of the patient's outcome, including risk of paralysis.

Exemplary ELISA kits are provided by StressXpress HSP70 and HSP27 ELISA kits (Stressgen, Vancouver, BC), using a mouse monoclonal antibody on pre-coated 96-well immunoassay plates. Standard negative controls and blocking steps are used in accordance with manufacturer's protocol. Human CSF from young healthy normal patients, and artificial CSF, both ‘spiked’ with known concentrations of human recombinant HSPs (multiple vendors) are used as positive controls. Assay performance milestones for the HSP assays include, but are not limited to:

1) assay results within 5% of known concentration in positive samples in human CSF;

2) lack of significant confounding effects of blood in CSF;

3) reproducibility less than 5% variation (measure of variation of repeated measures of same sample); and

4) stability of assay over time.

In the alternative, peptide or polypeptide concentrations can be measured by methods other than immunoassay. For example, the sample can be subjected to 2D-gel electrophoresis and the amount of the polypeptide estimated by densitometric scanning of the gel or of a blot therefrom

Because the shortest possible time to begin remediation measures is of the essence in correcting spinal cord ischemia and to prevent paresis or death, it is desirable to carry out the HSP assay in a rapid manner. A fiber-optic system from ForteBio, Inc., Menlo Park, Calif., offers rapid measurement of proteins in CSF, such as the HSPs, and its use is encompassed within the methods of the present invention. Other companies offer technology for rapid protein measurement using a variety of technologies, providing alternative rapid detection and quantitative measurement of HSP70 and/or HSP27 levels in CSF and blood. For example, full automation in a widely used clinical chemistry analyzer, such as the COBAS™ MIRA Plus system from Hoffmann-La Roche, described by Robers et al. Clin Chem. 44(7):1564-7 (1998) or the AxSYM™ system from Abbott Laboratories, is possible and can be applied for routine clinical diagnosis of spinal cord ischemia and related complications using the biomarker of the present invention.

The invention also relates to the use of one or more of the specified polypeptides which is differentially contained in the CSF of an aortic resection patient during surgery, as compared with the same patient (referred to as “within-patient”) prior to surgery or to a control sample, for diagnostic, prognostic and therapeutic applications. This invention may further involve the preparation and/or use of a material which recognizes, binds to or has some affinity to the HSP27 and/or HSP70 peptide or polypeptide. Examples of such materials are antibodies and antibody chips. The term “antibody,” as used herein, includes polyclonal antiserum, monoclonal antibodies, fragments of antibodies such as Fab, and genetically engineered antibodies. The antibodies may be chimeric or of a single species.

Included within the present invention are both manual and automated immuno-selective techniques that are presently available or as may be later developed that are effective for determining induced HSP levels in a CSF sample, or changed levels therein, as compared with another sample or with a control sample or predetermined profile. Methods that are used manually, may be automated to provide better or faster results, and are included within the present invention. For example, a re-usable 510k bench-top rapid ELISA device to monitor HSP70 and HSP27 concentrations in serial CSF samples collected from a patient undergoing TAA or TAAA repair is under development, in which clinical algorithms and statistical trends analyses, as provided herein, enable the measurements to be used to assess acute spinal cord function and ischemic damage, and to predict the risk of paralysis pre-operatively, intra-operatively, and post-operatively. Functioning in the operating room, preferable such device incorporates micro fluidics (e.g., 300-500 μl samples) to avoid the need to spin blood, with secondary antibody capture, possibly with a dedicated light or UV source and simple spectrophotometer for measurement. An on board small processor chip and automated control standards permits simple, automated calculations. Alternatively, samples may be sent to a separate lab, as currently used for arterial blood gases and routine chemistries.

Comparative Processing of the Data. Non-linear techniques for data analysis and information extraction are important for identifying the elevated presentation of a biomarker relevant to clinical outcome. However, variables such as a patient's prior history, physical condition, and location and extent of the aneurism, must also be included in the determinations. As controls, a “normal” population might also be used to establish baseline levels or confirm within-patient levels of the biomarkers.

While the biochemical identification of HSP27 and/or HSP70 proteins or polypeptides in the patient is not itself significant, the biomarker embodied in the present invention that determines whether there is a measurable intra-operative change in the level of those proteins in the patient's CSF during or following aortic surgery, establishes the probability or onset of spinal cord ischemia, and the possibility of patient paresis. Thus, simply identifying the presence of HSP27 and/or HSP70 in the patient's CSF may not necessarily be significant at all points in the patient's history, nor does the presence of HSP27 and/or HSP70 always predict, diagnosis or prognosis spinal cord ischemia or probable paralysis. Rather, the biomarker is the measurable, stress-induced change (or non-change) in the level of HSP27 and/or HSP70 in recurring CSF measurements during or as a direct result of the surgery in which aortic blood flow was stopped for a period of time.

For this purpose in one embodiment, data is presented as a spreadsheet, i.e., a written, printed or imaged two-dimensional table of values, with rows referring to time columns filled with patient biomarker and other characteristic values, such that the changed or elevated HSP27 and/or HSP70 concentration in the CSF of the patient can be charted or plotted with regard to time points when the samples were drawn. The numerical data, and/or the plots or charts created therefrom, provide tangible evidence of the changes indicating increased risk (or non-risk) to the patient. Reports may be further generated, as desired.

In another embodiment an algorithm is used to objectively select the most relevant data (“clinical inputs,” including the induced HSP27 and/or HSP70 levels as measured in the patient's CSF) for each time period that correspond to the outcome. When operated on a computer-based system, this process is also known as “feature selection.” Because the shortest possible time to begin remediation measures is of the essence in correcting spinal cord ischemia and to prevent paresis or death, rapid computations are made comprising the feature selection process, the ongoing data inputs from the measured induced levels of HSP in the patient's CSF, along with statistical representations of relevant clinical inputs, e.g., the patient characteristics referred to above (also referred to in the Example section as “primary exposure variables”). Data is recomputed with the introduction of each additional within-patient CSF sample collection, e.g., at different time points intra- and/or post-operatively. Thus, comparative computations rapidly identify any change or elevation in the induced biomarker levels, thereby differentiating and/or predicting spinal cord ischemia prognosis, diagnosis, and/or detection. The determination of a change (comparative value) in the measured HSP level in the CSF is a determination critical to the method of the present invention.

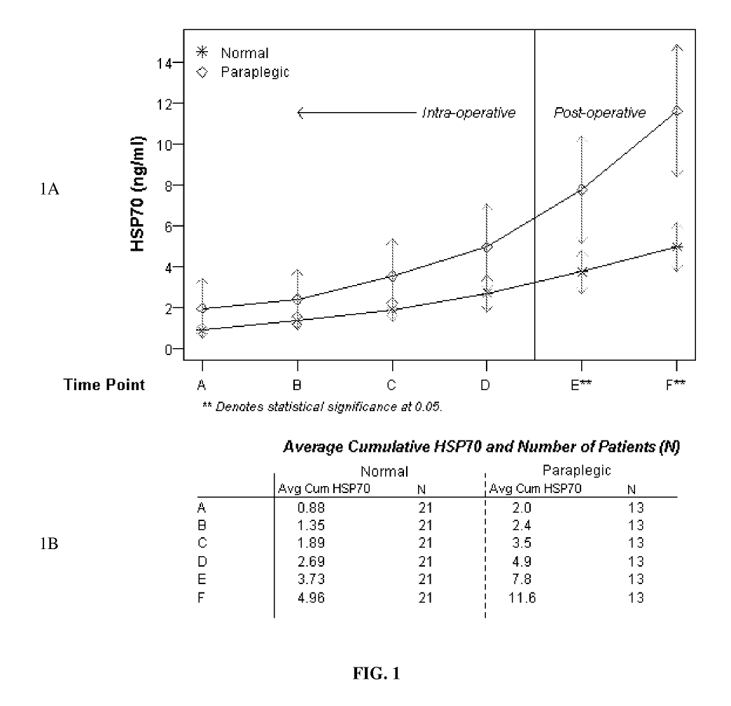

The data and clinical input is evaluated on a recurring basis with the introduction of sequentially collected data at the highest sensitivity and specificity, essentially establishing a time line corresponding to the HSP27 and/or HSP70 levels seen at each of the series of time points of the CSF measurements. See, FIGS. 1-3. The clinical sensitivity of an assay is defined as the percentage of those patients with the adverse event (e.g., spinal cord ischemia/paralysis) that the assay correctly predicts. The specificity of an assay is defined as comparison with the percentage of instances in which there is no adverse event that the assay correctly predicts (Tietz, Textbook of Clinical Chemistry, 2nd edition, Burtis & Ashwood eds., W. B. Saunders and Company, p. 496).

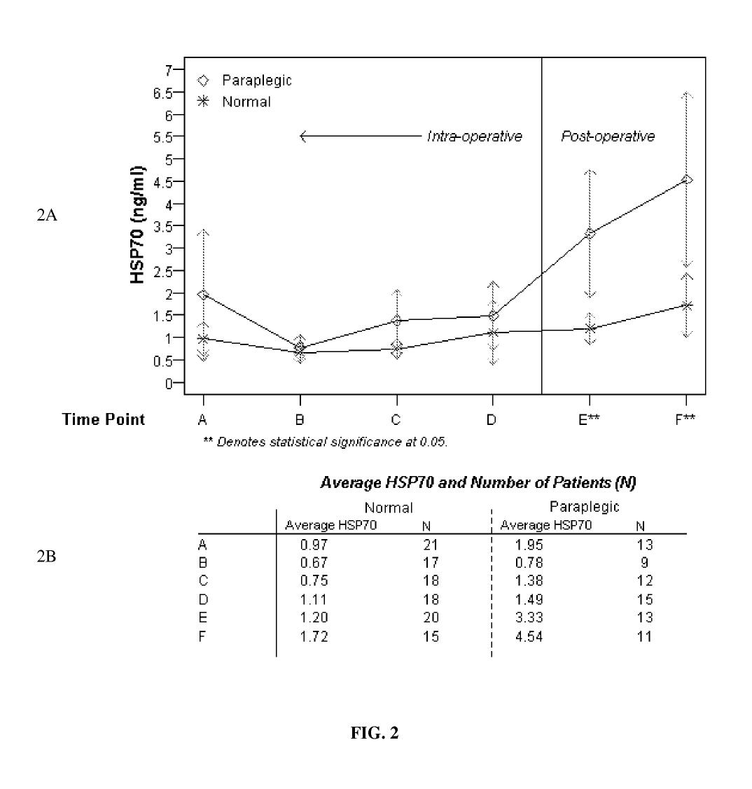

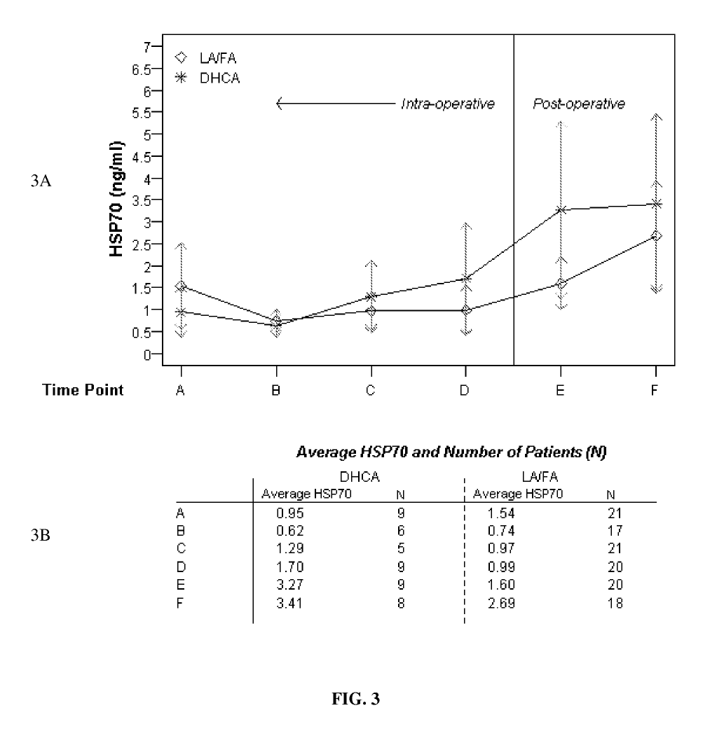

The feature selection may be done with an algorithm that selects the biomarker levels for intra-operatively induced HSP27 and/or HSP70, and differentiates those values (or a single measurement) with the within-patient baseline or control levels of the biomarker, preferably noting significant elevations in those levels in the patient's sampled CSF. The relevant clinical input combinations may change at different time periods, and might be different for different clinical outcomes of interest. As shown in FIGS. 1 and 2 respectively, the cumulative and average mean values of HSP70 concentrations levels were determined and plotted at each time point. Similar data was also collected for HSP27, showing similar effects. Further as shown in FIG. 3, the percentage of patients who developed subsequent paraplegia, versus those who did not, were compared as between DHCA surgery and LA/FA surgery, both considered to be open surgeries. Clinical input would be expected to change between patients. As a result, each patient's inputs and resulting output are computed separately.

Statistical analyses of the data in the present invention were computerized using commercially available statistical packages, e.g., as described in greater detail in the Example section that follows herein. Patient characteristics, including demographic characteristics and pre- and intra-surgical factors were also factored into the determinations. The “primary outcome” was the binary indicator of paralysis. To characterize the HSP27 and HSP79 concentration levels within the patient, “within-patient HSP27 and HSP70 values” were calculated using the summary statistics described in detail in Example section as primary exposure variables. Briefly, these included the slope of the within-patient linear regression fit to HSP27 and/or HSP70 values observed over time; the residual squared error (RSE) of the within-patient linear regression; maximum HSP27 and HSP70 concentration level values for each patient, and their range; as well as the measured and average ‘within-patient change’ and ‘percentage change’ as measured at times pre- and post-cross-clamp for HSP27 and HSP70 levels. See Table 3. Control data might also be used to establish baseline levels of the HSP in the CSF, as normal values for comparative purposes.

To confirm the strength of the embodied methods for determining predicted outcome, and differences between binary paralysis outcome groups were also characterized in the study presented in the Example section that follows. For discrete variables, the number of observations were computed in each level/outcome group combination, and tested for significant differences between groups with Fisher's exact tests. The Fisher exact test computes the difference between the data observed and the data expected, considering the given marginal and the assumptions of the model of independence. One of a class of exact tests, the significance of the deviation from a null hypothesis can be calculated exactly, rather than by relying on a test statistic having a distribution that is approximately that of a known theoretical distribution. In the present case, when samples are small in at least one of the cells, the exact test is a better choice than the Chi-square test of estimated probabilities in 2-by-2 tables. Thus, the probability is tested of getting a table as strong as the observed or stronger simply due to the chance of sampling, where ‘strong’ is defined by the proportion of cases on the diagonal with the most cases. Though usually employed as a one-tailed test, it may be computed as a two-tailed test as well. See, e.g., www.quantitativeskills (dot)com/sisa/statistics/fishrhlp.

For continuous variables, the medians and inter-quartile ranges were computed, and the nonparametric Wilcoxon rank sum test was used, as set forth in greater detail in the Example section, to assess significant differences between outcome groups.

A series of stepwise multivariable logistic regressions were used to evaluate the effects of the primary exposure variables on the paralysis outcome. Each of the primary exposure HSP27 and HSP70 variables was considered. The regression program (see Example section) mapped the selected relevant clinical inputs to the outputs. Such a regressor assigns relative “weightings” (or “weighting factors”) to individual biomarker level data input values, creating a comparative data set. The weighting factors are multiplicative of marker levels in a nonlinear fashion. Each weighting factor is a function of other input data in the comparative data set, and consists of terms that relate individual contributions, or independent and correlative, or dependent, terms. In the case of the biomarker having no interaction with other data in regards to then clinical outcome of interest, then the specific value of the dependent terms would be zero. A risk analysis was made on the basis of no paralysis or a prediction of no paralysis=0; while paralysis or a prediction of paralysis=1.

While the actual construction of a regressor/classifier is beyond the scope of the present methods of this particular invention, any comparative mapping procedure between inputs and outputs may be applied that produces a measure of ‘goodness of fit’ using the C Statistic, as exemplified in the Example section. Standard optimization routines on a series of validation sets would also suffice, e.g., to maximize the area under the receiver operator characteristic (ROC) curve of sensitivity versus 1-specificity.

Persons skilled in bioinformatics will recognize these procedures, and understand the availability of such algorithms, typically processed on a computerized system by known methods, to provide the statistical analyses needed to take advantage of the identified biomarkers in this invention for detection, prognostic, diagnostic and/or therapeutic purposes. The maximal sensitivity, specificity, and predictive power is realized by inputting the biomarker data into a data set containing other variables to constitute a group by a process of plotting receiver operator characteristic (ROC) curves for (1) the sensitivity of a particular data set, versus (2) specificity for the combination at various cutoff threshold levels.

Once the selected recurring biomarker data and collecting relevant clinical information for each new patient is entered, the regression application outputs a maximum likelihood estimator for the value of the output (see Table 3), given the inputs for the current patient. A cost function that the a classifier or regression program optimizes is specified according to the desired outcome, e.g., an area under the ROC curve, maximizing the product of sensitivity and specificity of the selected biomarker levels, or positive or negative predictive accuracy. The regressor maps input variables, in this case patient biomarker concentration values, to outcomes of interest, for instance, prediction of spinal cord ischemia and/or paresis. Preferred classifiers or regressors include, but are not limited to, neural networks, decision trees, genetic algorithms, SVMs, regression trees, cascade correlation, Group Method Data Handling (GMDH), Multivariate Adaptive Regression Splines (MARS), multilinear interpolation, radial basis functions, robust regression, cascade correlation+projection pursuit, linear regression, non-linear regression, polynomial regression, regression trees, multilinear interpolation, MARS, Bayes classifiers and networks, and Markov models, and kernel methods. Preferred methods for classifier optimization include, but are not limited to, boosting, bagging, entropy-based, and voting networks to provide a final predictive model.

Computations in a predictive model determine the expected clinical outcome by interpolating biomarker and other data input to predict or determine clinical outcome. While constructing a computerized predictor exceeds the scope of this invention, predictor models are commercially available. However, selection of the model is important, because errors at each step can propagate downstream, affecting the ability to generalize the certainty of the predicted clinical outcome.

In certain embodiments of the invention, data outputs relevant to the clinical outcome of interest are subjectively determined by the surgeon, neurologist, anesthesiologist or clinician (including technicians, nurses, and others working with the physician) in light of the immunologically, or otherwise determined levels of HSP27 and/or HSP70 induced in the patient's CSF. Such a predictive model can be translated into a decision tree format for subdividing the data and making the decision output of the model easy to understand for the clinician, particularly in terms of expected outcome with regard to comparative biomarker levels at aortic cross-clamping or release, and/or as compared with a previously established threshold value for average actual change values and/or average percentage of change values.

An alternative embodiment of the invention comprises a computer software program and algorithm comprising a computer-based predictive model that interprets the biomarker assay values and recurring measurements of elevated levels of stress-induced HSP27 and/or HSP70, preferably, but not always, beginning prior to surgery. In this case, the predictive model receives the marker values via the computer that it resides upon, or from a device that it is connected to, and provides computed clinical output. From the biomarker-based information, for example: (1) the output of interest may be a risk of paralysis, (2) the output of interest may be onset of permanent paralysis (=1), triggering immediate remediation steps by the surgical team, (3) the output of interest may indicate no onset of paralysis or a low risk of paralysis (=0), indicating to the surgical team a measure of safety for proceeding with the surgery. Thus, the data output may be a single numerical assessment of risk or probability of spinal cord ischemia and resulting patient paresis, or it may comprise an optimum range of output values, given the data inputs, measured in combination with clinical conditions, and specific threshold values if available.

While the damaging or fatal effect of spinal cord ischemia during aortic resection has long been know as a serious complication of such surgery, until the present invention, there was no intra-operative method for predicting the risk of, or onset of, such spinal cord ischemia in time to take corrective steps before the patient has suffered irreversible paralysis. The methodology of the present invention, however, provides a direct correlation with the defined biomarker, as evaluated by subjective comparisons, including simple spreadsheet-type comparisons, or as confirmed by objective computerized methods. In either case, output data is generated upon which a medical team can rapidly act to attenuate and correct the ischemia, and lessen or prevent resulting paresis in the patient. A straightforward extension of the invention provides an optimum range of output values given patient inputs, as well as specific threshold values for inputs. The novelty of this discovery remains, regardless of whether the biomarker levels are computed alone, or in combination with other techniques.

The analysis, as presented in the Example section that follows, clearly demonstrates a nonlinear relationship between within-patient, stress-induced HSP27 and/or HSP70 values and spinal cord ischemia and the potential for resulting permanent paralysis. In general, although variables are possible between patients, as stress-induced HSP27 and/or HSP70 levels become increasingly elevated in the surgical patient undergoing aortic resection, the greater the risk that the patient will experience paralysis as an outcome. Thus, the present method that for the first time offers such information during the operative process, thereby alerting the physician responsible for the patient of changes in the patient's condition, and offering an invaluable early warning if rapid intervention will remediate neural damage and paralysis before such complications are irreversible.

An embodiment of the present invention comprises a computerized device using the statistical trends analyses and algorithms utilized herein, and improvements thereon, for computing clinical outcome as rapidly as possible from patient CSF samples, preferably before an absolute threshold of onset of spinal cord ischemia and/or paralysis has been reached.

Detection and Diagnostic Kits. The instant invention further encompasses a detection kit comprising reagents, devices and instructions for performing assays. In one embodiment the invention provides a detection kit for detecting an elevated level of stress-induced HSP27 and/or HSP70 in a patient's CSF measured pre-, intra- or post-operatively as a biomarker indicative of a predicted risk of spinal cord ischemia and/or resulting risk of paresis associated with aortic surgery. Such kits comprise: (1) an immunosorbent for HSP27 and/or HSP70 comprising a primary antibody, and (2) an indicator reagent comprising secondary antibodies attached to a signal generating compound for HSP27 and/or HSP70, by which the presence and concentration of HSP27 and/or HSP70 in the CSF of a patient may be determined by color-based or quantification assay. The secondary antibodies can be specific for the biomarker or for a primary antibody in the immunosorbent. The immunosorbent preferably comprises anti-antibodies for the biomarkers bound to a support system, e.g., an ELISA kit.

In another embodiment the invention provides a diagnostic kit for detecting stress-induced elevation of a patient's selected HSP27 and/or HSP70 biomarkers indicative of a risk of spinal cord ischemia and/or resulting risk of paresis. The kit comprises (a) data from a control sample or panel of control samples, providing a comparative baseline of “normal” levels of HSP27 and/or HSP70 expected in the CSF of a non-surgical individual, having a relatively matched clinical history as the patient on which the kit will be used; (b) an immunoassay such as an ELISA or a rapid assay test kit comprising a secondary antibody specific for the biomarker attached to a signal-generating compound, which may be used to determine the concentration of the selected HSP27 and/or HSP70 biomarker in the CSF of the patient at each time points; and (c) instructions permitting the comparison of the respective HSP27 and/or HSP70 levels in recurring patient samples (b) with the control data in (a), wherein elevation of the biomarker levels in (b) is indicative of ischemic risk.

The reagents may also include ancillary agents such as buffering agents and protein stabilizing agents, e.g., polysaccharides and the like. The diagnostic kit may further include, as necessary, other members of the signal-producing system, e.g., enzyme and non-enzyme substrates, agents for reducing background interference in a test, agents to increase the signal, apparatus for conducting a test, calibration and standardization information or instructions, and the like.

Optionally the kits may contain one or more means for using information obtained from immunoassays performed to establish a biomarker panel to rule in, or out, certain diagnoses. Marker antibodies or antigens may be incorporated into immunoassay diagnostic kits, depending upon which marker auto-antibodies or antigens are being measured. A first container may include a composition comprising an antigen or antibody preparation. Both antibody and antigen preparations should preferably be provided in a suitable titrated form, with antigen concentrations and/or antibody titers given for easy reference in quantitative applications.

The kits may also include an immunodetection reagent or label for the detection of specific immunoreactions, if any, between the provided antigen and/or antibody, and the diagnostic sample. Suitable detection reagents are well known in the art, as exemplified by radioactive, enzymatic or otherwise chromogenic ligands, which are typically employed in association with the antigen and/or antibody, or in association with a second antibody having specificity for first antibody. Thus, the reaction is detected or quantified by means of detecting or quantifying the label. Immuno-detection reagents and processes suitable for application in connection with the novel methods of the present invention are generally well known in the art.

The reagents may also include ancillary agents such as buffering agents and protein stabilizing agents, e.g., polysaccharides and the like. The diagnostic kit may further include, as necessary, agents for reducing background interference in a test, agents for increasing signal, software and algorithms for combining and interpolating biomarker values to produce a prediction of clinical outcome of interest, apparatus for conducting a test, calibration curves and charts, standardization curves and charts, and the like.

Such kits are preferably disposable, comprising devices and reagents for the analysis of at least one test sample, and instructions for performing the assay. In the alternative, the kits may include sufficient materials for collecting and analyzing multiple samples, e.g., 20-30 samples from the same patient, permitting standardization. As used herein, “instructions” or “instructional material” includes a publication, a recording, a diagram, or any other medium of expression which can be used to communicate the usefulness of the composition of the invention for its designated use. The instructional material of the kit of the invention may, for example, be affixed to a container, which contains the composition or be shipped, together with a container which contains the composition. Alternatively, the instructional material may be shipped separately from the container with the intention that the instructional material and the kit be used cooperatively by the recipient.

In one embodiment, these kits preferably comprise software, in addition to the reagents and devices, for measuring one or more marker levels in a patient sample, and instructions for performing the assay. For example, the kits may contain a computer software program to be run on a computer or other means for converting biomarker level(s) to a prognosis. Such kits preferably contain sufficient reagents to perform one or more such determinations, and are standardized to run on an instrument used to analyze CSF samples, such as produced by Abbott Laboratories, or Roche Diagnostics, or Dade Behring.

The present invention is further described in the following examples. These examples are not to be construed as limiting the scope of the appended claims. It will be apparent to those skilled in the art that many modifications, both to materials and methods, may be practiced without departing from the purpose or narrowing the scope of this invention.

### EXAMPLES

In this study data was collected from patients admitted to the inventors' institution for TAAA repairs from 2003 through 2006, wherein lumbar CSF drains were inserted as part of the standard care. Each patient had granted permission after full disclosure. Demographic data were collected preoperatively. Formal NIH Stroke Scale (NIHSS) and lower extremity American Spinal Injury Association (ASIA) scales were performed by a certified neurologist, who was not blinded to procedure or outcome, at within-patient baseline and 12, 24, and 48 hours postoperatively and at discharge. In cases in which acute neurological changes were detected, the patients were subjected to further exam using the National Stroke Association online National Institutes of Health Stroke Scale (NIHSS) and American Spinal Injury Association (ASIA) exams during a detailed neurologic examination, along with appropriate neuroimaging to confirm the existence of spinal or brain ischemia, and to exclude peripheral nerve or muscle injury.

If an episode of spinal ischemia was detected intra- or post-operatively, standard protocols were followed to try to reverse ongoing ischemia (see, Cheung et al., Ann. Thorac. Surg. 74:413-419 (2002); Cheung et al., Ann. Thorac. Surg. 80:1280-1289 (2005); McGarvey et al., Neurocritical Care 6:35-39 (2007)). Briefly, remediation protocols involved pharmacologic elevation of the mean arterial pressure (MAP), increasing intravenous fluids, replacing the lumbar drain if it had been removed, and correcting CSF drainage from 8 to 12 mmHg.

All patients received a narcotic-based anesthetic with inhaled isoflurane in oxygen. For open TAAA, circulation management consisted of either distal aortic partial left heart bypass with core cooling to 32° C., left atrium to femoral artery partial bypass (LA/FA bypass), or a deep hypothermic technique utilizing full cardio-pulmonary bypass (CPB) via the left chest with an open proximal anastomosis. Left atrium to femoral artery (LA/FA) bypass was used for aneurysms or dissections that did not involve the aortic root or aortic arch (type B or descending). If the arch vessels or aortic root were involved, either primarily or in a retrograde manner from the descending aorta, then deep hypothermic circulatory arrest (DHCA) with cardiopulmonary bypass was performed, with retrograde cerebral perfusion. For LA/FA management bypass, flow rates averaged 2.5 liters/min, adjusted to achieve a target distal aortic perfusion pressure of at least 60 mmHg, while maintaining proximal aortic pressure of at least 90 mmHg.

Repairs involving the distal arch repair required utilization of DHCA, systemic cooling on CPB until the electroencephalogram (EEG) reached electrical cerebral silence (12-18° C.), then termination of CPB to perform the open proximal anastomosis with total body retrograde cerebral perfusion (300-500 cm/min at a central venous pressure of 12-15 mmHg) via an unsnared superior vena cava. After completion, arterial circulation was reinitiated via the proximal descending Dacron graft, the distal anastomosis was performed, and the patient was rewarmed. For combined distal arch and type II and III TAAA repairs, arterial circulation was reinitiated with both proximal and distal femoral artery perfusion.

Lumbar CSF drains were used in both LA/FA bypass and hypothermic cases. The spinal cord central pressure was maintained at 10-12 mmHg with lumbar CSF drainage, theoretically to maximize the perfusion pressure gradient between systemic circulation and spinal cord. The lumbar CSF drain was typically clamped 24 hours after surgery, and removed after 48 hours, absent neurological deficits. Intercostal arteries were reimplanted in all patients when possible.

Three patients in this cohort underwent descending thoracic aortic repair involving thoracic endovascular aortic repair (TEVAR), without CPB involving general anesthesia, femoral artery cannulation, angiography, and aneurysm exclusion using an aortic stent graft.

CSF sample collection. Serial CSF samples were collected as a part of the approved protocols at the following time points (to the extent possible):

(A) at lumbar drain placement immediately after anesthetizing and intubating the patient for surgery (this can be done with patient awake, but most commonly, particularly for TAAA or TAA patients, the lumbar drain is placed by the anesthesiologist shortly after induction of anesthesia, when the patient is asleep).

(B) at the time of placement of the aortic cross-clamp in cases involving LA/FA bypass, or at the time of restart of CPB after DHCA, or aortic stent deployment in TEVAR procedures (temporally collectively referred to as “post-clamp”);

(C) 1 hour after step (B), or in an alternative embodiment in actual surgery, samples are drawn every 20 minutes for the first four (4) hours after step (B);

(D) 2 hours after step (B), or in an alternative embodiment in actual surgery, (C) and (D) may be combined, and samples are drawn every 20-30 minutes after step (B) until the end of surgery;

(E) 12 hours after step (B); or in an alternative embodiment in actual surgery, post-operative samples are drawn every 2 hours over a total of 6 hours after the surgery has ended, and one more sample can be taken at 12 hours after step (B) if the CSF drain remains in place;

(F) 24 hours after step (B) if the CSF drain remains in place.

The identified time points represent blocks of time, not necessarily specific collections, although in the Example that follows, collections were made at each point defined above. However, multiple sample collects may be made within each block of time at, e.g., 5, 10, 15, 30, or 60 minute intervals, or at wider intervals of, e.g., 1 hour, 3 hours, 12 hours, 24 hours, etc. Further CSF samples were collected at various time points, and the times need not be uniformly spaced. Additional collection was made if there was any sign of the development of postoperatively paraparesis. Samples were not collected if the intracranial pressure (ICP) was not greater than 8 mmHg or if the lumbar drain was not functional. For diagnostic purposes, the samples are tested immediately.

For study purposes, test samples (including separate corresponding blood samples) were immediately centrifuged, and the supernatants, free of cell debris, were stored frozen at ˜80° C. Blood samples were collected and centrifuged, and the plasma aliquoted and stored in a process like that used for the CSF. Blood samples are saved for collaborative purposes and may be used for systemic testing of HSP, but are not part of the biomarker of this invention.

HSP70 and HSP27 concentrations in the patient's CSF samples were analyzed after surgery was finished, and after the post-operative outcomes of these patients were known. Patient results were divided into two subsets of patients: (1) patients with paraplegia (type 1a=“permanent,’ or type 1b which resolved with intervention=“transient”) and (2) patients with no signs or symptoms of paraplegia postoperatively=“normals”). Notation was made of patients with intraoperative changes in measured somato sensory-evoked potentials (SSEP), with or without paraplegia, and induced HSP levels were correlated with patient outcomes.

In the present study, statistical significance was affected by the initial data set, due to missing values (e.g., when samples had been consumed before completion of all tests), and sample standard deviation. The applied difference of means approach, implying pair wise comparison of two means, is a very conservative approach, given that both HSP70 and HSP27 are measured at each sample collection, and repeated measures of linked variables are made, i.e., assuming that the responses of an individual follow a trajectory over time.

ELISA assay for HSP70 and HSP27. HSP70 and HSP27 were quantitatively measured with StressXpress HSP70 and HSP27 ELISA kits (Stressgen, Vancouver, BC, Canada). These kits use a mouse monoclonal antibody on pre-coated 96-well immunoassay plates. HSP70 and HSP27 were captured by immobilized anti-bodies by loading 100 μl CSF/well in duplicate on the plates, followed by incubation at 4° C. overnight. After rinsing, captured HSP70 and HSP27 were probed by incubation at room temperature (RT) with a secondary biotinylated rabbit polyclonal anti-mouse antibody (1:500 dilution). Unbound secondary antibodies were removed by rinsing. Biotinylated antibodies were incubated at RT with an avidin-horseradish peroxidase conjugate diluted 1:500. After rinsing, the assay was developed by incubation with tetramethylbenzidine substrate and intensity was measured in a microplate reader (Dynex Technology) at 450 nm. Standard curves were plotted on a log-log scale versus absorbance. Sequential sample results for HSP27 and HSP70 were tabulated and analyzed.

Statistical methods: Prior to performing analyses, standard data screening/cleaning procedures were applied. These procedures screen for data entry errors, check for outliers, assess the extent and pattern of missing data, and check that appropriate assumptions of normality are met whenever necessary. Randomization was checked by comparing groups on relevant background variables, and variations on which groups show significant differences may be included as covariates in later analyses.

All statistical analyses used the commercially available statistical packages (Statistical Applications Software Version 9.1, SAS Institute, Cary, N.C.) and R (The R Project for Statistical Computing Version 2.6.0). Each hypothesis involves a two-group comparison on a continuous outcome. Assuming a significance level of 2.5% and within group standard deviations of 0.5, a total sample size of 80 will provide 90% power to detect a minimum between-group difference of 0.4. Secondary analyses applied significance levels of 5% for all tests. Generalized estimating equation (GEE) techniques were employed to perform longitudinal analyses. Power is determined by the within-subject correlations, the significance level, and the amount of available data. This approach allows a flexible correlation structure between measurements at different time points, more efficiently accounts for within patient variability, and allows more subjects with some missing time points to be included in analyses.

The “primary exposure variables” (also referred to herein as clinical input) were measurements of the levels of human CSF proteins HSP70 and HSP27 taken at up to ten time points per patient during the surgical procedure or postoperatively. Patient characteristics, including demographic characteristics and pre- and intra-surgical factors were also factored into the determinations. The “primary outcome” was the binary indicator of paralysis. To characterize the concentration levels within the patient, “within-patient HSP27 and HSP70 values” were calculated using the following summary statistics to serve as primary exposure variables:
The slope of the within-patient linear regression fit to HSP27 and HSP70 concentration level values observed over time;
The residual squared error (RSE) of the ‘within-patient’ linear regression, used to explore the linearity or non-linearity of HSP27 and HSP70 values as observed over time, as indicators of clinical outcome, specifically paralysis.
The maximum HSP27 and HSP70 concentration level values observed for each patient;
The ranges of the HSP27 and HSP70 concentration values observed for each patient;
The within-patient change and percentage change from time point (B) to time point (C) (i.e., pre- and post-cross-clamp—see time points stated in Example section) HSP27 and HSP70 values. Note that these changes are positive if the time point (C) value is higher than that of time point (B);
The within-patient change and percentage change from the average pre- and post-cross-clamp HSP27 and HSP70 values. Note that these changes are positive if the post-cross clamp average is higher than the pre-cross clamp average; and the pre-cross clamp averages include time points (A) and (B) if time to cross clamp is greater than 60 minutes, and post-cross clamp averages includes time points after cross-clamp.

Differences between binary paralysis outcome groups were characterized with respect to several demographic and surgical variables (patient characteristics) in addition to HSP27 and HSP70 variables listed above. By considering the extensive demographic and intra-operative data that was collected, ensured that groups are matched and significant variables with effects on the outcomes can be determined. Longitudinal analyses used mixed-effects regression models. The main advantages of these models are that they extended the traditional repeated-measures framework in several ways. They allowed for a more flexible correlation structure between measurements at different time points, for more within group variability, and for more subjects with some missing time points to be included in analyses.

These models assume that the responses of an individual follow a trajectory over time, such as a linear or quadratic curve. The parameters that determined the shape of an individual trajectory were regarded as responses in regression models, and covariate effects were assessed by using them as predictors in the regressions. Terms corresponding to the variables comprise the fixed effects. The most relevant variables appeared to be age, co-morbidities, extent of resection, and perhaps DHCA versus LA/FA, genomics, and unknowns such as vascular anatomy. Additional terms and random effects were included for each individual to model the correlation between observations on the same individual.

In terms of missing data, it was assumed that the missing data was missing at random, which is usually reasonable. In all analyses, the assumptions underlying the application of all statistical methods that were used were examined, principally through the use of standardized residuals, influence diagnostics, and graphical displays. For discrete variables, the number of observations were computed in each level/outcome group combination, and tested for significant differences between groups with Fisher's exact tests. For continuous variables, the medians and inter-quartile ranges were computed, and the nonparametric Wilcoxon rank sum test was used to assess significant differences between outcome groups. Wilcoxon, F., Biometrics Bulletin 1:80-83 (1945) offers an alternative to the paired Student's t-test when the population can not be assumed to be normally distributed. All p values represent two-sided hypothesis tests.

Mean and cumulative values of HSP27 and HSP70 levels were plotted at each time point to evaluate the relationship between changes in HSP levels measured in the CSF and post-operative paraplegia. Boxplots were used to depict the relationship between the binary paralysis outcome and patient demographic variables and the calculated within-patient HSP27 and HSP70 values.

Multivariable logistic regression: A series of stepwise logistic regressions are used to assess the strength of the relationship between HSP27 and HSP70 values, operating as the primary exposure variables, and the paralysis outcome. Each of the primary exposure HSP27 and HSP70 variables was considered in a separate model. Performance criteria for detection of spinal cord ischemia will include sensitivity to clinical outcome classification >90%, and specificity for clinical outcome classification >80%.

This method is further used to explore the potential confounding of the HSP70 and HSP27 effects with patient demographic and surgical variables, e.g., demographic effects of age race, gender, and smoking history, using the standard statistical multivariate analysis model. The criterion for the stepwise selection of variables was p<0.25. The C-Statistic was used to evaluate the goodness of fit of the models (C=0.80). Finally, generalized estimating equations are fit to further quantify the relationship between changes in HSP27 and HSP70 over time and the paraplegia outcome.

### Example 1

Demographic, preoperative, and surgical statistics for the study population (37 patients) are shown in Table 1. Patients ranged in age from 40 to 80, with 20 men and 17 women. None of the demographic variables of age, race, and sex was significantly associated with the paralysis outcome. However, patient smoking history was found to have a statistically significant effect on the paralysis outcome of the patients. Patients who developed postoperative paraplegia reported a median of 60 (inter-quartile range, 50 to 75) pack years, compared with only 7 (inter-quartile range, 0 to 45) pack years among patients who did not develop paraplegia (p=0.0011). There were significantly more patients with chronic renal insuffiency (CRI) in the paralysis outcome group (40%, standard error=26%) than in the normal outcome group (4.6%, standard error=4.7%).

Regarding Surgical Statistics (see Table 1), significantly fewer patients in the postoperative paralysis outcome group than in the normal outcome group underwent surgeries other than DHCA or LA/FA [0% versus 22.7% (standard error=8.9%)]. This “Other” category under Surgical Statistics on Table 1, included three TEVAR (stent) patients and two patients in which the repair used open surgical procedures that did not require either DHCA or cardiac bypass.

Of the 37 non-consecutive thoraco-abdominal aneurysm patients in this study, 13 showed large increases of induced HSP27 and HSP70 levels within 2 hours post-cross-clamping of the aorta, and 12 more had large increases within 48 hours post-cross-clamp. Of the 25 patients, 13 showed postoperative paraparesis of some degree. Twenty-two of the 25 patients, who had significant HSP increases, showed either intraoperative SSEP or EEG changes consistent with brain or spinal cord ischemia, or postoperative neurological evidence of paraplegia or stroke. Of the 12 patients without significant HSP increases, only one had any evidence of paraplegia or central ischemia.

TABLE 1
Correlation of HSP Levels and Probability of Post-operative Paralysis:
Demographic and Surgical Statistics for Study Population.

| | | |
|---|---|---|
| Paraplegia = Yes | Paraplegia = No | p value |

Population Statistics

| | | | | | |
|---|---|---|---|---|---|
| Number of Patients | 15 | | 22 | | |
| Age [Median (25%, 75%)] | 68 | (61, 79) | 66.5 | (56, 77) | NS |
| Race [% (SE)] | | | | | |
| African American [%(SE)] | 13.3% | (8.8%) | 31.8% | (9.9%) | NS |
| Caucasian | 80% | (10.3%) | 63.6% | (10.3%) | |
| Hispanic | 6.7% | (6.5%) | 4.6% | (4.7%) | |
| Sex [% (SE)] | | | | | |
| Male | 60% | (12.6%) | 59.1% | (10.5%) | NS |
| Female | 40% | (12.6%) | 40.9% | (10.5%) | |

Preoperative Statistics

| | | | | | |
|---|---|---|---|---|---|
| Number of Patients | 16 | | 21 | | |
| Pack Years [median (25%, 75%)] | 60 | (50, 75) | 7 | (0, 45) | 0.0019 |
| HTN [%(SE)] | 93.3% | (6.5%) | 90.9% | (6.1%) | NS |
| MI [% (SE)] | 26.7% | (11.4%) | 27.3% | (9.5%) | NS |
| COPD [% (SE)] | 26.7% | (11.4%) | 27.3% | (9.5%) | NS |
| DM [% (SE)] | 0% | (0.0%) | 18.2% | (8.2%) | NS |
| CRI [% (SE)] | 40% | (12.6%) | 4.6% | (4.5%) | 0.0113 |
| Afib [% (SE)] | 6.7% | (6.5%) | 9.1% | (6.1%) | NS |
| CAD [% (SE)] | 0% | (0.0%) | 18.2% | (9.2%) | NS |
| Stroke [% (SE)] | 26.7% | (11.4%) | 18.2% | (8.2%) | NS |
| CABG [% (SE)] | 6.7% | (6.5%) | 0% | (0.0%) | NS |
| Cholesterol [% (SE)] | 6.7% | (6.5%) | 13.6% | (7.3%) | NS |
| PUD [% (SE)] | 0% | (0.0%) | 0% | (0.0%) | NS |
| PVD [% (SE)] | 13.3% | (8.8%) | 9.1% | (6.1%) | NS |

Surgical Statistics

| | | | | | |
|---|---|---|---|---|---|
| Number of Patients | 16 | | 21 | | |
| Cardiopulmonary Bypass [% (SE)] | 33.3% | (12.2%) | 27.3% | (9.5%) | NS |
| Circ arrest [% (SE)] | 26.7% | (11.4%) | 27.3% | (9.5%) | NS |
| LAFA [% (SE)] | 73.3% | (11.4%) | 50% | (10.7%) | NS |
| Other [% (SE)] | 0% | (0.0%) | 22.7% | (8.9%) | 0.0673 |
| Bypass time [median (25%, 75%)] | 131 | (94, 176) | 85.5 | (0, 159) | 0.0288 |
| Circ arrest time [median (25%, 75%)] | 0 | (0, 14) | 0 | (0, 7) | NS |
| Cross-clamp time [median (25%, 75%)] | 65 | (59, 88) | 65.5 | (45, 79) | NS |
| Emergent presentations [median (25%, 75%)] | 0% | (0.0%) | 18.2% | (8.2%) | NS |
| Contained ruptures [% (SE)] | 6.7% | (6.5%) | 9.1% | (6.1%) | NS |
| Dissections [% (SE)] | 20% | (10.3%) | 13.6% | (7.3%) | NS |
| Stents [% (SE)] | 0% | (0.0%) | 13.6% | (7.4%) | NS |
| Active hypo [% (SE)] | 26.7% | (11.4%) | 27.3% | (9.5%) | NS |
| Reimplantation of intercostals [% (SE)] | 46.7% | (12.9%) | 40.9% | (10.5%) | NS |

The following definitions apply to the acronyms used in Table 1: hypertension (HTN); myocardial infarction (MI); chronic obstructive pulmonary disease (COPD); diabetes mellitus (DM); chronic renal insufficiency (CRI); atrial fibrillation (Afib); coronary artery disease (CAD); peptic ulcer disease (PUD); peripheral vascular disease (PVD); coronary artery bypass graft (CABG); left atrial femoral artery (LAFA).

Cumulative and average mean values of HSP27 and HSP70 were computed at each time point after induction of anesthesia. The cumulative averages of HSP70 (FIGS. 1A and 1B) and HSP27 levels from those patients who subsequently developed paraplegia were higher than those of the patients who did not develop paraplegia. Mean values for HSP70 were also higher at each intra- or post-operative time point (C) through (F) for paraplegics versus normals, respectively (FIGS. 2A and 2B). However, only HSP70 values at time points (E) and (F) were statistically significant in both FIGS. 1 and 2 in these sample sizes. Graphs for HSP27 were similar (not shown).

Mean HSP70 and HSP27 levels were plotted to compare surgical technique (LA/FA and DHCA) with changes in the HSP levels (see HSP70 levels in FIGS. 3A and 3B; similar results were seen for HSP27). The percentage of patients who developed paraplegia was comparable in both open surgical repair subgroups (4 of 10 DHCA patients, and 10 of 20 LA/FA patients). However, the mean HSP70 levels at successive time points in patients with paraplegia after DHCA were considerably higher, as compared with the patients with paraplegia after LA/FA, especially in the levels measured 12 and 24 hours post-cross-clamp (FIGS. 3A and 3B).

TABLE 2
Within-Patient HSP Statistics.

| | | |
|---|---|---|
| Paraplegia = yes | Paraplegia = no | |
| Median (25%, 75%) | Median 25%, 75%) | p value |

HSP70

| | | | | | |
|---|---|---|---|---|---|
| Number of patients | 15 | | 22 | | |
| Linear regression slope | 0.52 | (0.13, 1.04) | 0.20 | (0.03, 0.36) | 0.0946 |
| Linear regression residual squared | 1.92 | (1.11, 2.71) | 0.51 | (0.42, 0.79) | 0.0005 |
| error | | | | | |
| Maximum | 8.16 | (3.01, 9.19) | 1.80 | (1.13, 3.04) | 0.0001 |
| Range | 7.26 | (2.59, 8.97) | 1.60 | (0.93, 2.27) | <0.0001 |
| Change from B to C | 0.05 | (−0.08, 0.85) | 0.12 | (−0.31, 0.25) | NS |
| Percent change from B to C | 10.21 | (−20.26, 51.15) | 20.59 | (−37.63, 46.83) | NS |
| Average AB to C + change | 2.13 | (0.53, 3.45) | 0.39 | (−0.16, 0.77) | 0.0173 |
| Average AB to C + percent changed | 263.08 | (61.00, 306.75) | 70.35 | (−26.30, 127.64) | 0.0424 |

HSP27

| | | | | | |
|---|---|---|---|---|---|
| Number of patients | 13 | | 22 | | |
| Linear regression slope | 0.19 | (0.07, 0.39) | 0.06 | (0.02, 0.10) | 0.0187 |
| Linear regression residual squared | 0.61 | (0.37, 1.04) | 0.17 | (0.09, 0.35) | 0.0010 |
| error | | | | | |
| Maximum | 2.86 | (1.43, 3.36) | 0.54 | (0.34, 1.08) | 0.0006 |
| Range | 2.22 | (1.09, 3.12) | 0.48 | (0.19, 0.83) | 0.0012 |
| Change from B to C | 0.14 | (0.04, 1.01) | 0.00 | (−0.30, 0.02) | 0.0169 |
| Percent change from B to C | 50.09 | (11.41, 60.85) | −6.48 | (−30.60, 30.97) | 0.0290 |
| Average AB to C + change | 1.01 | (0.27, 1.39) | 0.12 | (0.00, 0.20) | 0.0072 |
| Average AB to C + percent changed | 172.48 | (47.41, 382.35) | 84.06 | (−4.30, 335.374) | NS |

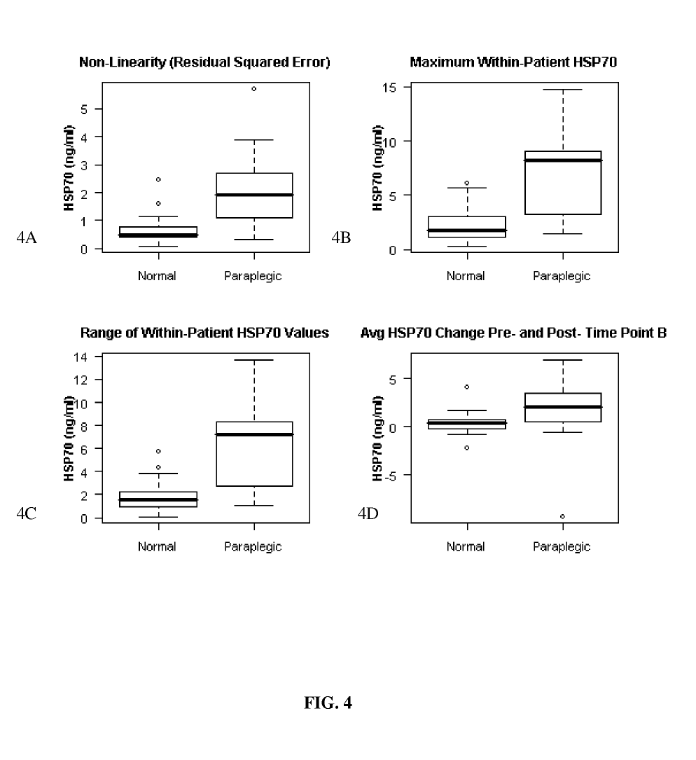

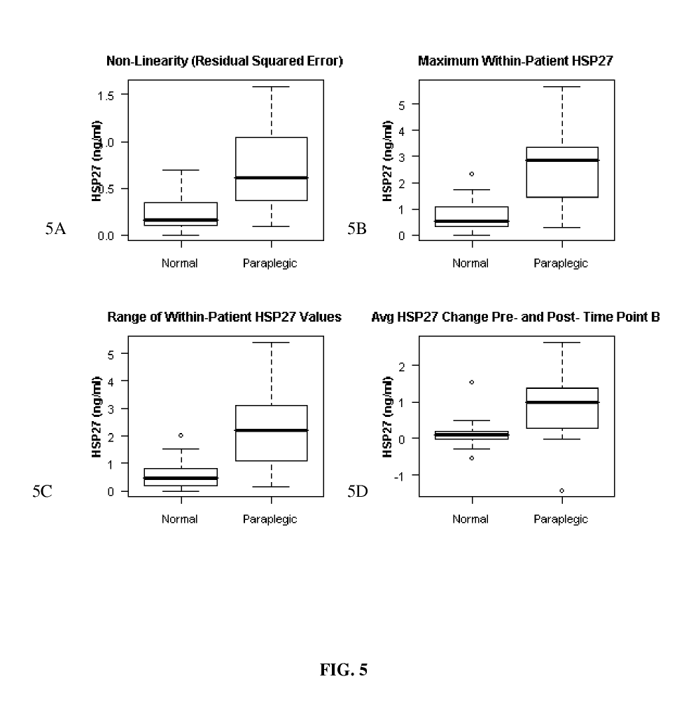

FIGS. 4 and 5 contain graphical and numerical comparisons of the paralysis and non-paralysis outcome groups with respect to each of the calculated variables listed above in Table 2. FIGS. 4 and 5 display side-by-side box-and-whisker plots of the independent variable distributions within the paralysis and non-paralysis groups for HSP70 and HSP27, respectively. In each box, the first quartile (Q1), median, and third quartile (Q3) are represented by the bottom of the box, thick line inside the box, and top of the box, respectively. “Whiskers” extend to the nearest data point within 1.5 times the inter-quartile range of the distribution. Individual points beyond the whiskers depict outliers. The p value resulting from a Kruskal-Wallis test of equal location parameters (i.e., medians) is given in Table 2. See, Kruskal and Wallis, J. Amer. Statistical Assoc. 47(260):583-621 (December 1952). Small p values (<0.05) indicate statistically significant differences between the two outcome groups.

Finally, the possibility was explored that these effects were confounded with basic patient demographic effects such as age, race, gender, and smoking history. Patient smoking history had a statistically significant effect on the paralysis outcome on the group of patients, wherein patients with a longer smoking history were more likely to experience paralysis. As expected, increasing age and emergency status also increased the risk for paraplegia, while race and gender did not impact outcome. Numerical summaries for the results are depicted graphically in FIGS. 4 and 5 as reported in Table 2.

Table 3, which lists effects with p values <0.10, summarizes the detailed logistic regression results. The results are further evidence that, for the study group of 37 patients, HSP70 and HSP27 values observed during surgery are indicative of paralysis. All significant effects were positive, meaning that patients with higher values of the calculated quantities were more likely to experience paralysis. More specifically, the patients with HSP70 and HSP27 measurements in CSF that were non-linear over time (i.e., higher RSE values), experienced more extremes (higher maximums and ranges), or had larger positive average changes from the pre- to post-cross-clamp time periods were more likely to experience paralysis than other patients.

TABLE 3
Multivariable Logistic Regression Results.

| | | |
|---|---|---|
| Primary exposure variable: | Odds ratio (95% conf. interval) | p value |

HSP70

| | | | |
|---|---|---|---|
| Linear regression slope | 2.852 | (0.523, 15.546) | NS |
| Linear regression residual | 5.574 | (1.538, 20.193) | 0.0089 |
| squared error | | | |
| Maximum | 1.870 | (1.202, 2.910) | 0.0055 |
| Range | 1.907 | (1.204, 3.019) | 0.0059 |
| Change from B to C | 2.455 | (0.515, 11.691) | NS |
| Percent change from B to C | 1.001 | (0.986, 1.016) | NS |
| Average AB to C + change | 1.149 | (0.843, 1.567) | NS |
| Average AB to C + percent | 1.002 | (0.999, 1.006) | NS |
| changed | | | |

HSP27

| | | | |
|---|---|---|---|
| Linear regression slope | 675.293 | (3.294, >999.999)^{a} | 0.0164 |
| Linear regression residual | >999.999 | (3.740, >999.999)^{a} | 0.0226 |
| squared error | | | |
| Maximum | 6.132 | (1.470, 25.568 | 0.0128 |
| Range | 6.038 | (1.451, 25.124 | 0.0134 |
| Change from B to C | >999.999 | (0.063, >999.999)^{a} | NS |
| Percent change from B to C | 1.028 | (0.999, 1.057) | 0.0617 |
| Average AB to C + change | 3.815 | (1.064, 13.67) | 0.0399 |
| Average AB to C + percent | 1.001 | (0.999, 1.002) | NS |
| changed | | | |

^{a}Large sample approximation to confidence interval. The >999 value for the odds ratio means that the estimate is unstable, mostly due to the small sample size. These types of models are iterative, and in order to settle on a “stable” point estimate, need to converge to a single value.

Patients with non-linear HSP27 and HSP70 changes over time, as seen in larger within-patient linear regression RSE values (Table 2), were more likely to experience paraplegia after surgery (p=0.0010 for HSP27 and p=0.0005 for HSP70). Larger maximum HSP27 measurements in CSF were observed in patients with postoperative paraplegia, with a median within-patient maximum HSP27 of 2.86 (inter-quartile range, 1.43 to 3.36) among paraplegics, compared with only 0.54 (inter-quartile range, 0.34 to 1.08) among non-paraplegics (p=0.0006).

A similar comparison can be made for HSP70 within-patient maximum values (p=0.0001). Wider within-patient HSP ranges were observed among patients who developed paraplegia than among patients with a normal postoperative outcome [median range of 7.26 (inter-quartile range, 2.59 to 8.97) versus 1.60 (0.93, 2.27) for HSP70, p<0.001; median range of 2.22 (inter-quartile range, 1.09 to 3.12) versus 0.48 (0.19, 0.83) for HSP27, p=0.0012)]. Patients with larger average HSP27 and HSP70 changes between the pre- and post-cross-clamp periods (time period (B)) were more likely to experience postoperative paraplegia. This was true both for average actual changes and for average percentage changes (see Table 2 for detailed results, including p values).

Stepwise multivariable logistic regression results provide further evidence that, for this representative study group of 37 patients, within-patient patterns of pre- and intraoperative HSP27 and HSP70 measurements in the CSF are indicative of paralysis, even when controlled for demographic and surgical variables (Table 3). In order to assess the association between each HSP27 and HSP70 independent variable and the paralysis outcome, a separate model was fit for each independent variable. Thus, a total of sixteen models were considered: eight for HSP27 and eight for HSP70. For each model, the outcome variable was the binary paralysis/no paralysis indicator. The smoking history variable (Pack Years) was also included as a potential confounder in each model. Consequently, stepwise regression methods were employed to determine the best-fitting final model.

In sum, it has here been verified for the first time that, even after adjustment for patient characteristics, patients with HSP concentration measurements that were non-linear over time (i.e., higher RSE values) were more likely to experience post-operative paraplegia (HSP70 odds ratio=5.574, 95% confidence interval 1.538 to 20.193, p=0.0089). It was also determined that patients with elevated pre- and intra-operative induced HSP levels in their CSF were more likely to experience paralysis [maximum HSP70 odds ratio=1.870 (95% CI: 1.202 to 2.910), p=0.0055; within-patient HSP70 range odds ratio=1.907 (95% CI: 1.204 to 3.019), p=0.0059]. CI refers to the standard term, confidence index. Finally, it was concluded that, even after adjusting for demographic, pre-operative, and intra-operative variables, higher elevations in average induced HSP27 levels from the pre- to post-cross-clamp time periods were indicative of paralysis [time point (B) to (C) percent change odds ratio=1.028 (95% CI: 0.999 to 1.057), p=0.0617; average AB to C+change odds ratio=3.815 (95% CI: 1.064 to 13.67), p=0.0399].

The disclosure of each patent, patent application and publication cited or described in this document is hereby incorporated herein by reference, in its entirety.

While the foregoing specification has been described with regard to certain preferred embodiments, and many details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Such modifications and additional embodiments are also intended to fall within the scope of the appended claims.

We claim:

1. A method for predicting or detecting onset of neurological ischemia intra-operatively in a patient undergoing thoracic aortic surgery, the method comprising:
collecting two or more samples of cerebral spinal fluid (CSF) from the patient at sequential intervals, wherein the first sample is collected at a pre-operative time point, at the start of surgery, or at a time point early in the surgery before clamping of the aorta; and wherein a second sample is, or subsequent samples are, collected at intra-operative time points;
analyzing each CSF sample by a rapid detection and measurement assay, using reagents specific for detecting and differentially quantifying a heat shock protein (HSP), thereby quantifying the concentration level of the HSP in each CSF sample at the time the sample was collected;
comparing the quantified HSP level in the second sample, or the quantified HSP level in each subsequent sample, with the quantified HSP level in the first sample, or in the case of more than one subsequent CSF samples, comparing the quantified HSP level in each sample with the quantified HSP level in the one or more samples taken at time points before it;
determining change in the compared levels of HSP in the CSF samples; and
correlating the determined change as a biomarker for predicting or detecting onset of neurological ischemia, wherein degree of elevation correlates with a heightened risk of ischemia and/or of patient paralysis.

2. The method of claim 1, wherein the HSP is HSP27 or HSP70, or both HSP27 and HSP70 measured in the same CSF sample.

3. The method of claim 1 or 2, wherein the neurological ischemia comprises spinal cord or brain ischemia.

4. The method of any one of claims 1-3, further comprising correlating detecting elevated levels of HSP27 or HSP70, or levels of both, with risk of permanent post-operative patient paralysis.

5. The method of any one of claims 1-4, wherein the detection and measurement assay comprises an enzymatic and/or immunoselection assay.

6. The method any one of claims 1-5, wherein immunoselection assay is specific for HSP27 and/or HSP70, and wherein the assay is an enzyme-linked immunosorbent assay (ELISA).

7. The method of any one of claims 1-5, wherein the rapid detection and measurement assay for HSP27 and/or HSP70 comprises fiberoptics.

8. The method of any one of claims 1-7, wherein absence of elevation of quantified HSP level in one or more CSF samples at intra-operative time points comprises a biomarker indicating a low risk of neurological ischemia and/or low risk of permanent patient paralysis as a result of the surgery.

9. The method of any one of claims 1-8, wherein the thoracic aorta surgery comprises repair of a thoraco-abdominal aneurysm (TAAA) or of a thoracic aneurysm (TAA).

10. The method of any one of claims 1-9, further comprising considering clinical patient information as a factor in determining the presence of a heightened risk of ischemia and/or permanent patient paralysis.

11. The method of any one of claims 1-10, wherein a high level of HSP27 and/or HSP70 in the patient's CSF at a pre-operative time point, as compared to a predetermined control value, comprises a biomarker indicating a need to modify or cancel the patient's surgery to avoid or minimize risk of post-operative patient paralysis.

12. The method of any one of claims 1-11, wherein the biomarker elevation in HSP27 and/or HSP70 levels between two or more CSF samples provides a warning of heightened risk of neurological ischemia and/or patient paralysis indicating a need for medical intervention to attenuate or reverse neurological ischemia before an absolute threshold of onset of ischemia and/or paralysis is reached.

13. The method of any one of claims 1-12, further comprising correlating the detected elevation of HSP27 and/or HSP70 with intra-operative somatosensory evoked potential (SSEP) measurements in the patient.

14. A method for predicting permanent post-operative patient paralysis, the method comprising:
extending collecting and analyzing samples of the patient's CSF to one or more post-operative time points; comparing the differentially quantified HSP level in each post-operative sample with the quantified HSP level in the one or more samples taken at time points before it;
determining change in the compared levels of HSP in the CSF samples; and
correlating the determined change as a biomarker for predicting risk of post-operative patient paralysis, wherein degree of elevation correlates with a heightened risk of post-operative permanent patient paralysis.

15. The method of claim 14, further comprising considering clinical patient information as a factor in determining the presence of a heightened risk of post-operative permanent patient paralysis.

16. The method of claim 14 or claim 15, wherein the post-operative biomarker elevation in HSP27 and/or HSP70 levels between two or more CSF samples provides a warning of heightened risk of post-operative patient paralysis indicating a need for medical intervention to attenuate or reverse the neurological ischemia before an absolute threshold of onset of permanent paralysis is reached.

17. The method of any one of claims 1-16, further comprising a recurrence of the analyzing, comparing, determining, and correlating steps as each additional CSF sample is collected from the patient.

18. The biomarker in accordance with any one of claims 1-17.

19. The method of any one of claims 1-17, further comprising using a computer-based system using statistical trends analyses and algorithms for rapidly computing clinical outcome from quantified biomarker changes in HSP levels patient CSF samples, and a predictive algorithm for prognosis or diagnosis of risk or onset of neurological ischemia and/or permanent patient paralysis, wherein each algorithm comprises a capability to map (i) the HSP expression levels and non-proteomic values as input data, (ii) clinical patient data, and (iii) historical clinical results, as output data; and to conduct an automated procedure to vary the mapping function, inputs to outputs relevant, in combination, to output clinical results predictive or diagnostic of risk or onset of intra- or post-operative neurological ischemia and/or permanent patient paralysis.

20. The method of claim 19, wherein the correlating step is performed in accordance with an algorithm drawn from the group comprising: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; probability function algorithms; recursive feature elimination or entropy-based recursive feature elimination algorithms; a plurality of algorithms arranged in a committee network; and forward floating search or backward floating search algorithms.

21. A kit comprising devices and HSP-specific reagents for the analysis of at least one CSF sample collected in any one of claims 1-17, and instructions for performing the assay.

22. The kit of claim 21, further supplied with devices and HSP-specific reagents for the analysis of 10-30 within patient CSF sample, and instructions for performing and standardizing multiple assays.
